(19) United States
(12) Patent Application Publication
(10) Pub. No.: US 2026/0290591 A1
(43) Pub. Date: Sep. 24, 2026

(54) SYSTEM

(71) Applicant: SoftBank Group Corp., Tokyo (JP)

(72) Inventor: Mikiya MARUYAMA, Tokyo (JP)

(73) Assignee: SoftBank Group Corp., Tokyo (JP)

(21) Appl. No.: 19/557,210

(22) Filed: Mar. 5, 2026

(30) Foreign Application Priority Data
Mar. 19, 2025 (JP) 2025-044470

(51) Int. Cl. G16H 50/20; G06N 3/0475; G06N 3/0499; G06V 10/74; G06V 10/77; G16H 10/60; G16H 30/40

(52) U.S. Cl. CPC G16H 50/20; G06N 3/0475; G06N 3/0499; G06V 10/761; G06V 10/7715; G16H 10/60; G16H 30/40

(57) ABSTRACT

A system includes a processor that is configured to receive health condition information from a user, generate a first prompt for instructing a generative artificial intelligence model to analyze the received health condition information and to diagnose one or more possible disease names based on the analysis, and generate a second prompt for instructing the generative artificial intelligence model to evaluate a severity of each of the diagnosed disease names by referring to a database that stores severity information related to diseases, and to determine the severity based on the evaluation.

### CROSS-REFERENCE TO RELATED APPLICATION

This application is based on and claims priority under 35 USC 119 from Japanese Patent Application No. 2025-044470 filed on Mar. 19, 2025, the disclosure of which is incorporated by reference herein.

### BACKGROUND

### Technical Field

The present disclosure relates to a system.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

Conventional medical triage and preliminary diagnosis systems that rely on rule-based engines or manually designed decision trees are limited in their ability to flexibly interpret diverse, free-form health condition information provided by users. In particular, such systems often cannot adequately analyze complex or ambiguous symptom descriptions, cannot easily be adapted to new diseases or medical knowledge, and provide only coarse or static severity assessments. As a result, users may receive inaccurate or insufficient guidance regarding possible diseases and their severity, which can delay appropriate medical consultation or treatment. Furthermore, existing systems generally do not seamlessly integrate generative artificial intelligence models into end-to-end workflows that include severity evaluation, medication selection in cooperation with nearby medical facilities, and recommendation of appropriate medical institutions. Therefore, there is a need for a system that utilizes generative artificial intelligence models in a structured and controllable manner, by generating prompts that instruct such models to perform analysis of user health condition information, diagnosis of possible disease names, severity evaluation by referring to a database, selection and provision of medications in cooperation with nearby medical facilities, and recommendation of suitable medical institutions, thereby improving the quality, timeliness, and personalization of medical guidance provided to users.

### SUMMARY

In order to solve the above-described problems, according to one aspect of the present invention, there is provided a system comprising a processor, wherein the processor is configured to receive health condition information from a user, generate a first prompt for instructing a generative artificial intelligence model to analyze the received health condition information and to diagnose one or more possible disease names based on the analysis, and generate a second prompt for instructing the generative artificial intelligence model to evaluate a severity of each of the diagnosed disease names by referring to a database that stores severity information related to diseases, and to determine the severity based on the evaluation. The processor may further be configured to generate a third prompt for instructing the generative artificial intelligence model to cooperate with one or more nearby medical facilities, to select one or more appropriate medications based on the diagnosed disease names and the determined severity, and to provide the selected medications. The processor may further be configured to generate a fourth prompt for instructing the generative artificial intelligence model to recommend one or more appropriate medical facilities based on an analysis result obtained from the received health condition information, and to generate a recommendation message including information on the one or more appropriate medical facilities. By structurally defining and generating prompts that control the generative artificial intelligence model in these ways, the system enables flexible and accurate diagnosis of possible diseases, dynamic severity evaluation using a database, coordinated selection and provision of medications, and context-aware recommendation of medical institutions, thereby achieving improved medical support for users.

The term “system” refers to an integrated combination of hardware and software components, including at least one processor and associated memory, communication interfaces, and programs, that are configured to perform one or more of the processes described in the present specification and claims.

The term “processor” refers to any hardware component or set of components capable of executing instructions, including but not limited to a central processing unit (CPU), a graphics processing unit (GPU), a digital signal processor (DSP), a microcontroller, an application specific integrated circuit (ASIC), a field programmable gate array (FPGA), or any combination thereof.

The term “health condition information” refers to information related to a physical or mental condition of a user, including but not limited to symptom descriptions, body temperature, duration and onset of symptoms, medical history, chronic diseases, current medications, demographic information, and any other data relevant to a medical or health assessment.

The term “user” refers to a human individual who provides health condition information to the system and receives diagnosis, severity evaluation, medication guidance, or medical facility recommendations from the system.

The term “generative artificial intelligence model” refers to a machine learning model configured to generate output data, such as text, structured data, or other content, in response to input data and instructions, and includes, for example, large language models, multimodal generative models, and other neural network-based models capable of generating diagnostic analyses, severity evaluations, medication selections, or recommendation messages.

The term “prompt” refers to data that is supplied to a generative artificial intelligence model to specify or constrain a task to be performed by the model, and may include instructions, queries, templates, system messages, user messages, context information, and parameters that together define how the model analyzes input data and generates output.

The term “first prompt” refers to a prompt generated by the processor to instruct the generative artificial intelligence model to analyze the received health condition information and to diagnose one or more possible disease names based on the analysis.

The term “second prompt” refers to a prompt generated by the processor to instruct the generative artificial intelligence model to evaluate a severity of each of the diagnosed disease names by referring to a database that stores severity information related to diseases, and to determine the severity based on the evaluation.

The term “third prompt” refers to a prompt generated by the processor to instruct the generative artificial intelligence model to cooperate with one or more nearby medical facilities, to select one or more appropriate medications based on the diagnosed disease names and the determined severity, and to provide the selected medications.

The term “fourth prompt” refers to a prompt generated by the processor to instruct the generative artificial intelligence model to recommend one or more appropriate medical facilities based on an analysis result obtained from the received health condition information, and to generate a recommendation message including information on the one or more appropriate medical facilities.

The term “diagnose” refers to the act performed by or via the generative artificial intelligence model of identifying one or more possible disease names or medical conditions that may correspond to the health condition information provided by the user.

The term “possible disease name” refers to a candidate disease or medical condition identified by the generative artificial intelligence model as potentially corresponding to the health condition information, and may be associated with additional data such as a confidence score or probability.

The term “severity” refers to an assessment of the seriousness or urgency of a disease or medical condition, and may include, for example, categorization into levels such as mild, moderate, or severe, or into any other gradation indicative of risk or required medical attention.

The term “database” refers to any data storage system, including relational databases, key-value stores, document stores, or other structured or semi-structured data repositories, that stores information related to diseases, their severity, medications, or medical facilities, and that is accessible by the processor for evaluation or reference.

The term “nearby medical facilities” refers to hospitals, clinics, pharmacies, or other medical or healthcare institutions that are located within a certain distance from a location associated with the user, such as a current location or registered address, and that may be identified or selected by the system for cooperation or recommendation.

The term “medication” refers to any pharmaceutical product, including prescription drugs, over-the-counter drugs, or other therapeutics, that is selected or recommended by the system in response to the diagnosed disease names and the determined severity.

The term “medical facilities” refers to institutions that provide medical or healthcare services, including but not limited to hospitals, clinics, emergency centers, specialized medical centers, and partner institutions capable of prescribing or dispensing medications or providing medical consultations.

The term “recommendation message” refers to information generated by or via the generative artificial intelligence model that indicates one or more appropriate medical facilities or medications, and may include details such as facility names, addresses, departments, contact information, and reasons for recommendation.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### First Exemplary Embodiment

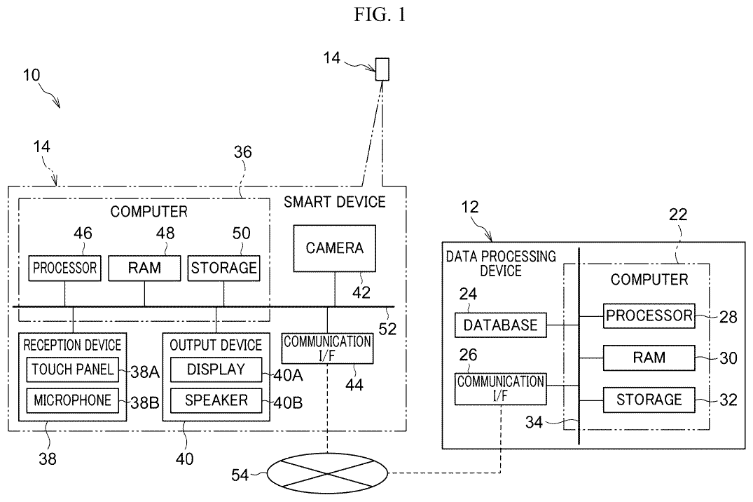

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

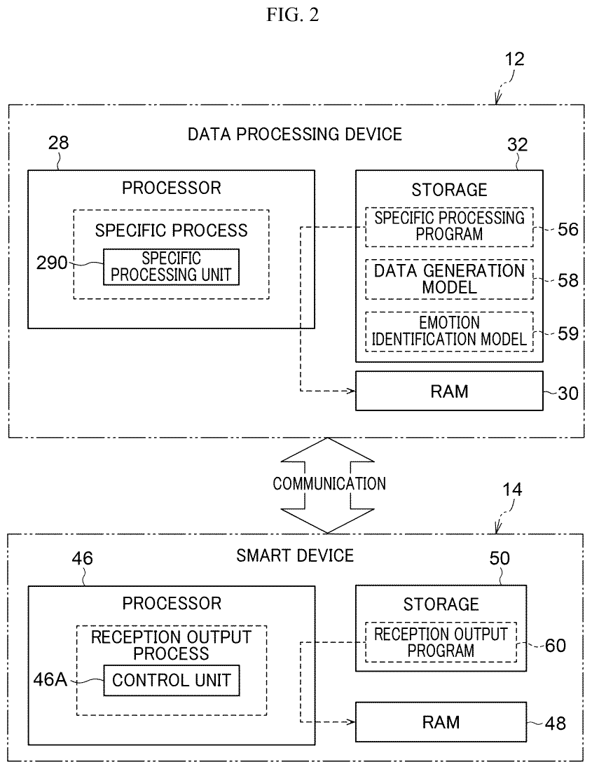

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

Conventional computer-implemented medical support systems that attempt to assist users in understanding their health status based on self-reported information typically rely on fixed rule sets, pre-defined decision trees, or narrowly scoped machine learning models. In such systems, a processor usually applies rigid, hand-crafted rules to map a limited set of symptom codes to a limited set of disease codes and severity levels. Because the mapping logic is static and tightly coupled to specific input formats, these systems have difficulty handling free-form natural language descriptions of symptoms, combining heterogeneous data such as symptoms, body temperature, and medical history, and adapting to new medical knowledge without extensive manual reprogramming.

Furthermore, in many existing architectures, natural language understanding and subsequent severity evaluation are implemented as separate, loosely integrated components. For example, a first component may attempt to recognize disease names from user text, while a second component separately queries a database for severity. The processor often has to perform ad-hoc string matching or post-processing on unstructured text responses, which leads to high latency, inconsistent outputs, and fragile error handling. As a result, the overall system suffers from reduced reliability, difficulty in scaling to large vocabularies, and increased computational overhead due to repeated parsing and heuristic processing.

In addition, when a generative AI model is used in a naive way, the processor typically sends an underspecified prompt to the generative AI model and receives a purely narrative, unstructured text response. The processor then has to perform complex natural language parsing and heuristic extraction of disease names and explanations on the server side. This post-processing is computationally inefficient, prone to misinterpretation, and difficult to maintain, thereby degrading the performance and robustness of the computer system as a whole.

Moreover, existing systems often lack a mechanism by which the processor can systematically combine AI-generated candidate disease names with structured severity information from a database, and then compute an overall severity classification in a deterministic and machine-efficient manner. The absence of a well-defined data flow from user input, through prompt construction and AI inference, to database lookup and final recommendation, results in fragmented architectures with duplicated logic, inconsistent severity assessments, and increased implementation complexity.

Accordingly, there is a need for a computer-implemented technique that improves the operation of the computer system itself by: (i) converting heterogeneous health status information from a user terminal into structured data suitable for efficient processing; (ii) generating a controlled prompt sentence that guides a generative AI model to output candidate disease names and explanations in a machine-readable format; (iii) integrating that output with a structured information storage device storing severity information; and (iv) deterministically computing and transmitting diagnosis result information, including overall severity and recommendation information, to the user terminal with improved reliability, scalability, and computational efficiency. The technical problem to be solved is to provide an improved server-side processing architecture and data flow that reduces ad-hoc natural language post-processing, enhances the consistency of severity determination, and thereby improves the functioning of the underlying computer system that supports medical guidance.

The specific processing by the specific processing unit 290 of the data processing device 12 in Example 1 is realized by the following means.

The present invention provides a server comprising a processor configured to receive health status information from a user terminal, convert the health status information into structured data, and store the structured data; generate a prompt sentence described in natural language based on the structured data including information on symptoms, body temperature, and medical history, the prompt sentence including a structured output condition that instructs a generative AI model to output candidate disease names and explanation information in a machine-readable format; input the prompt sentence into the generative AI model so as to cause the generative AI model to analyze the structured data and to output the candidate disease names and the explanation information in the machine-readable format; parse the candidate disease names and the explanation information output in the machine-readable format to extract the candidate disease names; refer to an information storage device that stores severity information associated with disease names, acquire the severity information for each of the candidate disease names, and determine overall severity based on the candidate disease names and the severity information; and generate diagnosis result information including the candidate disease names, the severity information, the explanation information, and recommendation information, and transmit the diagnosis result information to the user terminal. This enables the server-side computer system to execute a structured, end-to-end data processing pipeline that minimizes ad-hoc natural language post-processing, leverages the generative AI model as a controllable component within a deterministic architecture, and efficiently integrates AI-derived candidate disease names with database-stored severity information, thereby improving reliability, scalability, and computational performance of health status assessment and recommendation processing.

The term “system” refers to a collection of one or more information processing devices, storage devices, and communication interfaces that cooperate to execute the functions recited in the claims.

The term “processor” refers to one or more hardware processing units, such as a central processing unit, a graphics processing unit, or any other arithmetic and logic execution unit, configured by executable instructions to perform operations on data.

The term “user terminal” refers to an information processing device operated by a user, such as a mobile device, a portable computer, a stationary computer, or any other computing apparatus capable of inputting, transmitting, and displaying information.

The term “health status information” refers to information related to a physical or mental condition of a user, including, for example, symptom information, body temperature information, and medical history information.

The term “structured data” refers to data represented in a predefined, machine-readable format, such as a record, an object, or a hierarchical data structure, in which items such as symptoms, body temperature, and medical history are associated with respective fields or keys.

The term “prompt sentence” refers to a sequence of one or more natural language expressions or tokens, generated by the processor, that is provided as input to a generative AI model to cause the generative AI model to perform an analysis or generation task.

The term “generative AI model” refers to an information processing model, such as a neural network model, configured to receive input data including a prompt sentence, perform natural language processing, and output generated information such as candidate disease names and explanation information.

The term “machine-readable format” refers to a data representation format that can be directly parsed and processed by a computer program, such as a structured text format, a markup language, or a data serialization format.

The term “candidate disease name” refers to an identifier for a potential medical condition or illness that is inferred from the health status information by the generative AI model or other analysis.

The term “explanation information” refers to information indicating a reasoning, basis, or rationale for associating a candidate disease name with the health status information.

The term “information storage device” refers to a storage apparatus or memory subsystem, such as a non-volatile storage device, a volatile memory device, or a database system, configured to store and provide data such as severity information or medical service provider information.

The term “severity information” refers to information indicating a degree or level of seriousness of a disease, including, for example, a severity classification, an urgency level, or recommended actions.

The term “overall severity” refers to an aggregated or synthesized indication of seriousness determined by the processor based on a plurality of candidate disease names and corresponding severity information.

The term “diagnosis result information” refers to information generated by the processor that includes at least a candidate disease name and associated severity information, and may further include explanation information and recommendation information.

The term “recommendation information” refers to information that suggests an action or response for a user in view of candidate disease names and severity information, including, for example, guidance to seek medical care or to perform self-care.

The term “medical history” refers to information relating to past or existing health conditions of a user, such as chronic diseases, prior diagnoses, prior treatments, or risk factors.

The term “medical service provider” refers to an entity that provides medical or health-related services, such as a healthcare institution, a clinic, a hospital, or another medical facility or practitioner.

The term “structured output condition” refers to an instruction included in the prompt sentence that defines a required output format for the generative AI model, such that the output is in a machine-readable format suitable for deterministic parsing by the processor.

In one embodiment, a server cooperates with one or more terminals operated by users to implement the claimed system. The server includes at least one processor, a main memory, a non-volatile storage device, and one or more communication interfaces. The processor may be implemented by a central processing unit, a graphics processing unit, or any combination thereof. The memory and the non-volatile storage device store executable instructions and data structures that configure the processor to perform the functions described in the claims. The communication interfaces enable the server to exchange data with the terminals over a communication network such as the Internet.

A terminal is a computing apparatus operated by a user, such as a smartphone, a tablet device, a laptop, or a desktop computer. The terminal includes an input interface, such as a touch panel or keyboard, a display device, a local processor, a memory, and a communication interface. The terminal executes an application program that provides a graphical user interface for the user to input health status information and to receive and display diagnosis result information from the server.

A user operates the terminal to input health status information describing a current condition. The user inputs free-form natural language text describing symptoms, such as “I have a headache and feel fatigued,” numerical values for body temperature, such as “37.8,” and textual or selectable information representing medical history, such as “hypertension” or “diabetes.” The terminal converts the user inputs into structured data by mapping individual pieces of information to respective fields in a data record, such as “symptoms,” “body_temperature,” “underlying_conditions,” and “timestamp.” The terminal may store this structured data in a local buffer in memory and transmit the data to the server in a serialized format such as a hierarchical key-value structure over a secure communication channel.

The server receives the structured health status information from the terminal and stores the information in the memory. The server uses an application layer program to parse the received data and to normalize the information into an internal data structure. For example, the server may convert body temperature values into a unified unit, tokenize symptom text into word tokens using natural language preprocessing routines, and normalize medical history terms using a local dictionary stored in the non-volatile storage device. By structuring and normalizing the data at the server side, the server reduces ambiguity and prepares the data for efficient processing by a generative AI model and a severity evaluation module.

The server generates a prompt sentence in natural language based on the structured data. The server uses a prompt construction module implemented as executable instructions that perform deterministic string operations on the structured data. The server inserts specific field values, such as the normalized symptom description, the unified body temperature value, and the standardized medical history terms, into a prompt template. For example, the server may generate a prompt sentence such as:

“User's symptoms: headache and fatigue. Body temperature: 37.8° C. Underlying conditions: hypertension. Based on the user's symptoms, body temperature, and underlying conditions, please diagnose possible disease names and briefly explain the reasoning.”

In another example, the server may generate a prompt sentence that explicitly specifies an output structure, such as:

“User's symptoms: sore throat and cough. Body temperature: 38.2° C. Underlying conditions: asthma. Based on the user's symptoms, body temperature, and underlying conditions, please diagnose possible disease names. Output the result in a machine-readable structure with fields ‘possible_diseases’ as a list of names and ‘explanation’ as a textual explanation.”

By generating such prompt sentences, the server constrains the behavior of the generative AI model and enforces a consistent, machine-readable output format. This prompt-based structuring differs from conventional rule-based medical expert systems and reduces the need for ad-hoc parsing of unstructured text.

The server uses a generative AI model implemented as a neural network architecture, such as a multi-layer transformer network. The generative AI model includes an embedding layer that maps discrete tokens to continuous vectors, a plurality of self-attention layers that compute attention scores between tokens, and feed-forward layers that transform contextualized token representations. The generative AI model has been trained in advance using a large corpus of textual data and fine-tuned with health-related texts. During training, the model minimizes a loss function such as a cross-entropy loss between predicted token sequences and reference sequences. The model updates internal parameters, such as weight matrices of attention heads and feed-forward networks, using an optimization algorithm such as stochastic gradient descent with adaptive learning rate. The model may also employ regularization techniques such as dropout and data augmentation on text sequences during training.

The server inputs the prompt sentence into the generative AI model by converting the prompt sentence into tokens using a tokenizer, mapping the tokens into embeddings, and performing forward propagation through the transformer layers to compute output token probabilities. The generative AI model generates output tokens in an auto-regressive manner based on a decoding strategy such as greedy decoding, beam search, or sampling. The server configures generation parameters, such as maximum output length, temperature, and top-k or top-p thresholds, to control the diversity and determinism of the generated output. The server obtains a text output that includes candidate disease names and explanation information, expressed according to the structured output condition indicated in the prompt sentence. The server parses the output of the generative AI model using a parser module. When the prompt sentence instructs the model to output data in a structured form, the parser module operates as a deterministic state machine that recognizes specific markers and field labels in the output and maps them to corresponding internal data fields. For example, when the generative AI model outputs:
“possible_diseases: common cold; tension headache explanation: The user's symptoms and mild fever suggest a common cold or a tension-type headache.”
the server parses the string, extracts the list of candidate disease names “common cold” and “tension headache,” and stores them in an array data structure in memory. The server also extracts the explanation string and associates it with the array in the internal data record. By having the generative AI model produce a constrained, semi-structured output, the server can avoid complex natural language understanding pipelines and reduce computational overhead for post-processing.

The server refers to an information storage device that stores severity information associated with a plurality of disease names. The information storage device may be implemented as a relational database management system, a key-value store, or another database structure that maintains records mapping disease identifiers to severity classifications, urgency indicators, and recommended actions. For example, a database table may contain fields such as “disease_name,” “severity_level,” “urgency_level,” and “standard_recommendation.” The server executes a series of database queries to retrieve severity information for each candidate disease name obtained from the generative AI model.

The server determines an overall severity indication by aggregating the severity information of individual candidate disease names according to predetermined rules stored in the non-volatile storage device. For example, the server may implement a rule that if any candidate disease name has a “severe” severity level or a “high” urgency level, then the overall severity is set to “high,” whereas if all candidate disease names are “mild” and “low” urgency, then the overall severity is set to “low.” The server may store these aggregation rules as a rule table or as logical expressions that operate on severity and urgency codes. This rule-based aggregation improves consistency of severity judgment compared with ad-hoc, purely narrative outputs and reduces ambiguity in system behavior.

The server generates diagnosis result information by combining the candidate disease names, the severity information, the overall severity, the explanation information from the generative AI model, and recommendation information derived from the database. The server constructs a diagnosis result record that includes, for example, a summary statement, a list of candidate disease names with associated severity levels and urgency levels, a plain-language explanation, and a recommended action such as “monitor at home,” “schedule an outpatient visit,” or “seek immediate emergency care.” The server stores this diagnosis result record in memory and then transmits the record to the terminal via the communication interface.

The terminal receives the diagnosis result information and presents it to the user through the display device. The terminal may perform additional formatting, such as highlighting high severity conditions, displaying color codes, or sorting the list of candidate diseases by severity. The user reads the displayed information and may choose to follow the recommended action. The terminal may also store the diagnosis result information locally for later reference or forward it to other systems as allowed by configuration.

The server thereby improves the operation of the computer system in several technical aspects. First, by converting heterogeneous and unstructured health status information into structured data and generating highly constrained prompt sentences, the server reduces the amount of unstructured text that needs to be interpreted by local post-processing routines. This reduction leads to lower processing time in the parsing module and reduces memory consumption because the data stored and processed are compact and structured. Second, by instructing the generative AI model to output in a machine-readable format, the server eliminates multiple intermediate natural language processing stages that would otherwise be required to extract disease names from long narrative text. This architectural design reduces latency and increases throughput when multiple diagnosis requests are processed in parallel. Third, by integrating the generative AI model with a structured severity database and a deterministic aggregation module, the server creates a reproducible and testable data flow that mitigates non-determinism of free-form text generation. The server can easily validate severity outputs against predefined rules and update database entries without retraining the generative AI model. This modular separation between free-form reasoning (performed inside the generative AI model) and deterministic severity aggregation (performed by the server logic) results in improved maintainability, testability, and reliability of the system. Fourth, the server uses AI processing not merely to replicate human expert reasoning, but to leverage high-dimensional contextual representations learned by the transformer network. The attention layers in the generative AI model can consider long-range dependencies between symptom descriptions, body temperature values, and medical history terms. For example, the model can weight the co-occurrence of “chest pain,” “shortness of breath,” and “history of heart disease” more strongly than isolated symptoms, based on attention weights. This capability allows the system to capture subtle interactions that would be difficult to encode in traditional rule-based systems and yields improved diagnostic coverage and accuracy. Fifth, the server is capable of adjusting decoding parameters and prompt patterns in real time to achieve a desired balance between diversity and determinism. For instance, in high-severity domains, the server can configure the generative AI model to use low temperature and deterministic decoding to reduce variation in outputs, thereby improving the stability of severity estimates. In less critical cases, the server can allow more exploratory outputs. This dynamic control of generative behavior based on structured rules is not available in conventional static systems and contributes to higher computational efficiency and better resource utilization.

In another embodiment, the server may maintain multiple prompt templates optimized for different categories of health status information or user profiles. The server selects an appropriate template according to metadata such as the user's age group, geographic region, or device type. For example, for pediatric users, the server may generate a prompt sentence such as:

“User's symptoms: fever and rash. Body temperature: 39.0° C. Underlying conditions: none known. The user is a child. Based on the child's symptoms, body temperature, and underlying conditions, please diagnose possible disease names and explain any urgent warning signs.” By adapting the prompt templates and aggregation rules, the server can achieve specialized behavior without rewriting core domain logic, thereby improving extensibility and reducing implementation effort.

In a further embodiment, the server may use cached intermediate results, such as mappings from specific symptom combinations to frequent candidate disease sets, to reduce repeated workload on the generative AI model. When the server detects that incoming structured data match a previously processed pattern within a defined similarity threshold, the server may retrieve a stored list of candidate disease names as a starting point and use the generative AI model to refine or confirm the list. This hybrid approach reduces average inference time and lowers communication load between the server and external AI infrastructure.

In still another embodiment, the server may employ a local pre-filtering module that uses lightweight machine learning models, such as gradient-boosted trees or shallow neural networks, trained on structured symptom features, to estimate a coarse-grained risk category before invoking the generative AI model. When the risk category is extremely low and matches a stable pattern in the severity database, the server can skip or simplify interaction with the generative AI model and respond directly using stored templates. This selective invocation pattern further reduces processing time and computational cost, while reserving full generative processing for ambiguous or higher-risk inputs.

The described embodiments illustrate how the server, the terminal, and the user cooperate to implement the claimed system in a concrete, technically grounded manner. The server executes specific data transformations, prompt sentence generation algorithms, neural network inference, structured parsing, database lookups, and severity aggregation rules that collectively improve the performance and reliability of health status assessment. The terminal provides a structured interface for data input and output, and the user benefits from timely and consistent diagnosis result information. By organizing processing around structured prompts and machine-readable outputs, and by integrating a generative AI model within a deterministic data flow, the system achieves technical effects including improved processing speed, reduced post-processing complexity, higher consistency of severity judgments, and better scalability in computer resources.

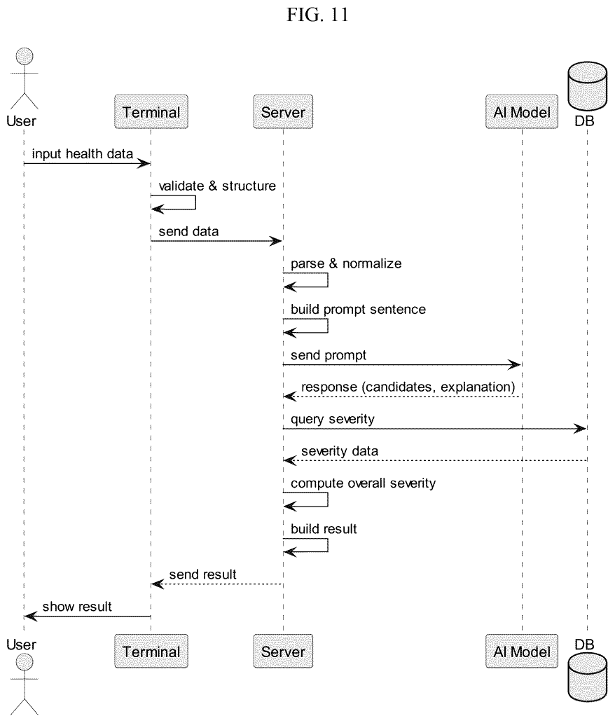

The following describes the processing flow using FIG. 11.

### Step 1:

The user operates the terminal to input health status information. The user provides, as input, free-form text describing symptoms, a numerical value for body temperature, and textual selections for medical history. The terminal receives these raw inputs from the user interface and converts them into internal data fields, for example mapping the symptom text to a “symptoms” field, the numerical value to a “body_temperature” field, and the medical history to an “underlying_conditions” field. The terminal outputs a structured record in memory that contains these fields as a coherent data object.

### Step 2:

The terminal validates and normalizes the structured record. The input to this step is the structured record created in Step 1. The terminal checks data types and ranges, for example verifying that the body temperature is within a medically reasonable interval and that mandatory fields such as symptoms are not empty. The terminal may also normalize formats, such as converting a temperature string into a floating-point number. Based on these checks, the terminal either prompts the user to correct invalid data or outputs a validated and normalized structured record ready for transmission.

### Step 3:

The terminal transmits the validated structured record to the server. The input to this step is the validated structured record from Step 2. The terminal serializes this record into a machine-readable transfer format and encapsulates it in a network request. The terminal then uses its communication interface to send the request through a communication network to a predefined server address. The terminal outputs a network message containing the serialized health status data as the request payload.

### Step 4:

The server receives and parses the health status data from the terminal. The input to this step is the network message sent by the terminal in Step 3. The server's communication interface accepts the message and passes the payload to an application process. The server deserializes the payload into an internal data structure, for example a record with fields for symptoms, body temperature, and underlying conditions. During this parsing, the server performs data conversion operations, such as decoding text into a unified character encoding and reconstructing numeric values from the serialized form. The server outputs a normalized health status data structure stored in server memory.

### Step 5:

The server performs additional normalization and feature preparation on the health status data. The input is the normalized health status data structure from Step 4. The server may tokenize the symptom text into word tokens, map medical history terms to standardized codes, and convert body temperature into a consistent unit if needed. The server may compute auxiliary features, such as a binary flag for fever based on a temperature threshold or a risk factor index based on the number and type of underlying conditions. The server outputs an enriched data structure that combines the original health status fields with derived features, ready to be embedded into a prompt sentence.

### Step 6:

The server generates a prompt sentence for the generative AI model based on the enriched data structure. The input to this step is the enriched data structure from Step 5. The server selects a prompt template and inserts specific field values, such as symptom description, body temperature, and underlying conditions, into predefined positions within the template. The server may also insert explicit instructions for output format and reasoning. For example, the server may generate a prompt sentence such as:

“User's symptoms: headache and fatigue. Body temperature: 37.8° C. Underlying conditions: hypertension. Based on the user's symptoms, body temperature, and underlying conditions, please diagnose possible disease names and briefly explain the reasoning.”

The server outputs a complete prompt sentence in natural language, represented as a sequence of characters or tokens in memory.

### Step 7:

The server augments the prompt sentence with a structured output condition. The input is the natural language prompt sentence from Step 6. The server appends or embeds instructions that require the generative AI model to return results in a machine-readable format, such as listing possible diseases under a specific label and providing an explanation under another label. For example, the server may transform the prompt into:

“User's symptoms: sore throat and cough. Body temperature: 38.2° C. Underlying conditions: asthma. Based on the user's symptoms, body temperature, and underlying conditions, please diagnose possible disease names. Output the result in a machine-readable structure with fields ‘possible_diseases’ as a list of names and ‘explanation’ as a textual explanation.”

The server outputs a modified prompt sentence that encodes both the medical context and the output formatting constraints.

### Step 8:

The server submits the modified prompt sentence to the generative AI model and obtains a generated response. The input is the modified prompt sentence from Step 7. The server converts the prompt sentence into tokens using a tokenizer, arranges them in a sequence, and forwards the token sequence to the generative AI model. Internally, the generative AI model applies embedding lookups, multiple self-attention and feed-forward layers, and a decoding algorithm to compute probability distributions over output tokens. Based on these distributions, the generative AI model produces a sequence of output tokens that represent candidate disease names and explanation information in the constrained format. The server reconstructs the output text from the tokens and outputs a generated response string that contains the candidate disease names and explanation information in a machine-readable layout.

### Step 9:

The server parses the generated response to extract candidate disease names and explanation information. The input is the generated response string from Step 8. The server applies a parsing routine that searches for field labels and delimiters, splits the response string into segments, and maps each segment to a corresponding internal field. For example, the server may identify a segment following “possible_diseases:” as a list of diseases and a segment following “explanation:” as explanatory text. The parsing routine may perform trimming, splitting on separators, and normalization of disease names. The server outputs a data structure that contains a list of candidate disease names and an associated explanation string.

### Step 10:

The server queries a severity database using the candidate disease names. The input is the list of candidate disease names from Step 9. The server constructs one or more database queries, each query specifying a candidate disease name as a lookup key. The server sends these queries to an information storage device that maintains a mapping between disease names and severity information. The database engine retrieves records that include severity level, urgency level, and recommended actions for each disease name. The server receives these records and organizes them into an array or table that aligns each candidate disease name with its corresponding severity information. The server outputs a combined structure containing candidate disease names and their retrieved severity information.

### Step 11:

The server computes an overall severity level based on the combined structure. The input is the combined structure of candidate disease names and severity information from Step 10. The server applies aggregation rules, such as scanning through all entries to find the maximum severity or highest urgency, and then mapping that maximum to a defined overall severity category. The server may also compute summary indicators, such as the number of severe conditions or the highest urgency code. By performing these comparisons and mappings, the server obtains a single overall severity level representing the worst plausible scenario among the candidate diseases. The server outputs the overall severity level and may store it together with the candidate disease data.

### Step 12:

The server generates diagnosis result information for the user. The input is the list of candidate disease names, the corresponding severity information, the overall severity level, and the explanation information from previous steps. The server assembles these data elements into a result record by performing string concatenation, field formatting, and arrangement into a structured representation. The server may create a summary sentence that describes the likely conditions and overall severity, followed by a detailed list of each candidate disease with its specific severity and urgency. The server may also convert recommended actions from the database into user-readable guidance. The server outputs a diagnosis result record that is ready for transmission to the terminal.

### Step 13:

The server transmits the diagnosis result record to the terminal. The input is the diagnosis result record from Step 12. The server serializes the record into a format suitable for network transmission and encapsulates it in a network response message. The server sends this message through the communication interface to the originating terminal. The server may log the outcome of the transmission and update internal state to reflect that the request has been processed. The server outputs a network response containing the diagnosis result information as its payload.

### Step 14:

The terminal receives and decodes the diagnosis result information. The input is the network response message sent by the server in Step 13. The terminal reads the response payload via its communication interface and deserializes the payload into an internal data structure. The terminal then extracts fields such as the overall severity, the list of candidate disease names with severity and urgency, the explanation text, and recommended actions. The terminal may perform minor formatting operations, such as sorting or truncating long text, to prepare the data for display. The terminal outputs a display-ready representation of the diagnosis result information.

### Step 15:

The terminal presents the diagnosis result information to the user. The input is the display-ready representation from Step 14. The terminal renders the overall severity level using appropriate visual indicators, such as color coding or icons, and lists candidate diseases along with their severity levels and urgency. The terminal displays the explanation text and recommended actions on the screen in a readable layout. The terminal may also store a local copy of the diagnosis result for future reference. The terminal outputs visual information to the user via the display device, enabling the user to understand the assessment produced by the system.

### Step 16:

The user reviews the diagnosis result information and may provide additional or updated health status information. The input is the visual information presented in Step 15. Based on the displayed severity and explanations, the user may decide to refine symptom descriptions, add newly observed symptoms, or correct prior inputs. The user operates the terminal's input interface to enter this new information. The terminal receives the updated inputs and again converts them into a structured record, similar to Step 1. The terminal outputs a new or updated structured health status record, which can be processed by repeating the subsequent steps of the flow.

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

In conventional computer-implemented medical advisory systems, a server typically receives health-related information from a user and performs rule-based or single-model statistical evaluation to output a tentative disease name or a simple triage level. However, such systems suffer from several technical limitations.

First, the server generally applies a monolithic inference model or static rule set to raw or lightly processed input data. As a result, the server fails to exploit the complementary strengths of different types of machine learning models, such as pattern-recognition models and language-generation models. This leads to suboptimal utilization of computing resources and reduces the flexibility and scalability of the system when handling diverse user inputs expressed in natural language.

Second, the server often couples diagnostic inference with user presentation in an ad hoc manner. The diagnostic output is typically a fixed code or short label, and any explanation or payment-related guidance is generated by simple template substitution. This rigid coupling prevents the server from dynamically adapting presentation detail or structure to the user's specific health condition or financial situation, and increases the need for manual configuration. As a consequence, the computing system exhibits poor adaptability and weak separation of concerns between diagnostic computation and user-facing explanation.

Third, in existing architectures, the server rarely treats prompt generation for generative AI models as a first-class computational function. Prompt text is either hard-coded or minimally parameterized, and the server does not systematically construct prompt sentences based on structured intermediate results, such as normalized health features, predicted disease severity, or stored payment-plan rules. This limits the accuracy and consistency of downstream generative AI processing, and can result in unstable or non-reproducible outputs, thereby degrading the reliability of the overall system.

Fourth, current systems do not integrate diagnostic outputs with personalized medical expense payment plans in a technically coherent way. The server may perform diagnosis and payment-plan selection as separate processes, without a unified data representation or automated mapping from disease severity to payment-plan parameters. This fragmented design increases computational overhead, complicates data management, and makes it difficult to maintain or extend the system as models and business rules evolve.

Accordingly, there is a need for a computer-implemented technique in which a server preprocesses user health information into structured formats, generates prompt sentences tailored for specific generative AI models, integrates analysis results from different models, and automatically maps disease names and severities to appropriate medical expense payment plans. By organizing the flow of data and computations in this manner, the system can improve the efficiency, modularity, and reliability of health-related decision support and payment-plan recommendation in a way that constitutes an improvement in the functioning of the computer system itself.

The specific processing by the specific processing unit 290 of the data processing device 12 in Application Example 1 is realized by the following means.

The present invention provides a server comprising a processor configured to receive, from a user terminal, information relating to a health condition of a user; to preprocess the information relating to the health condition into a predetermined data format including normalized numerical data and structured character data; to generate, on the basis of the preprocessed data, a first prompt sentence configured for input to an analysis generative AI model; to supply the first prompt sentence to the analysis generative AI model and obtain an analysis result including at least one disease name candidate and a corresponding severity; to integrate the analysis result with a prediction result obtained from a prediction learning model stored in an information storage unit, thereby determining a final disease name and a final severity; to refer to an information storage unit storing medical expense payment plan data in association with severity levels and to extract, on the basis of the final severity, a medical expense payment plan applicable to the user; to generate, on the basis of the final disease name, the final severity, and the extracted medical expense payment plan, a second prompt sentence configured for input to an explanation generative AI model; to obtain, from the explanation generative AI model, an explanation text regarding the medical expense payment plan; and to generate and transmit to the user terminal payment plan presentation information including the extracted medical expense payment plan and the explanation text. This enables the computer system to perform a structured sequence of data transformations and model invocations that improves the technical functioning of the server by enhancing the accuracy and stability of AI-based diagnosis, by optimizing the interaction between multiple machine learning models through prompt sentence generation, and by automatically coupling diagnostic outputs with individualized medical expense payment plans in a modular and scalable manner.

The term “processor” refers to a hardware or virtual computation unit, such as a central processing unit or a processing core, that executes instructions of a program to perform data processing, control, and communication operations within the system.

The term “user” refers to a person or entity that provides health-related information and receives diagnostic and payment-plan information through a user terminal.

The term “user terminal” refers to an information processing apparatus, such as a mobile communication device, a portable computing device, or a stationary computing device, that includes an input interface and a display interface and is configured to transmit data to and receive data from the server.

The term “information relating to a health condition” refers to data indicative of a physical or mental state of a user, including symptom descriptions, physiological measurements, medical history, and other health-related attributes.

The term “preprocess” refers to a series of operations performed on raw data, including formatting, normalization, cleaning, encoding, and transformation into a structured representation suitable for input to one or more machine learning models.

The term “numerical data” refers to health-related information expressed as numerical values, such as body temperature, heart rate, blood pressure, or duration of symptoms.

The term “character data” refers to health-related information expressed as text, including natural language descriptions of symptoms, conditions, or medical history.

The term “predetermined data format” refers to a structured representation of data defined in advance, such as a set of normalized numerical vectors, encoded categorical values, and tokenized text representations, that is suitable for processing by a machine learning model.

The term “prompt sentence” refers to a sequence of characters or tokens constituting an instruction or query that is supplied as input to a generative AI model to control or guide generation of output data.

The term “analysis generative AI model” refers to a generative artificial intelligence model that receives a prompt sentence relating to health information and performs language-based reasoning to output an analysis result including at least one candidate disease name and an associated severity.

The term “explanation generative AI model” refers to a generative artificial intelligence model that receives a prompt sentence relating to a diagnosis and a medical expense payment plan and outputs an explanation text suitable for presentation to a user.

The term “analysis result” refers to output data obtained from an analysis generative AI model, including at least one candidate disease name, an associated severity, and optionally explanatory information.

The term “disease name” refers to a designation of a medical condition or disorder used to identify a particular type of illness in a clinical or diagnostic context.

The term “severity” refers to an indication of the seriousness or intensity of a disease, represented by a qualitative level, such as mild, moderate, or severe, or by a quantitative scale.

The term “prediction learning model” refers to a trained machine learning model, such as a statistical classifier or neural network, that receives structured health-related input data and outputs a disease estimation result including at least one disease candidate and associated probability or severity information.

The term “disease estimation result” refers to an output of the prediction learning model indicating one or more candidate diseases and related scores or severities inferred from the input data.

The term “information storage unit” refers to a logical or physical data storage resource, such as a memory device or a database system, that stores data including model parameters, mapping tables, and medical expense payment plan data.

The term “medical expense payment plan” refers to data indicating a structure for payment of medical expenses, including at least a total amount, a division into installments or single payment, timing of payments, and optional adjustment conditions.

The term “medical expense payment plan data” refers to information stored in the information storage unit that defines one or more medical expense payment plans and associates such plans with disease severities or other criteria.

The term “severity level” refers to a classification category corresponding to a range or tier of severity, used to map a disease condition to one or more medical expense payment plans.

The term “extract” refers to an operation in which the processor selects one or more data items, such as a medical expense payment plan, from data stored in the information storage unit according to specified conditions or criteria.

The term “payment plan presentation information” refers to data generated for transmission to the user terminal and configured to present to the user at least one medical expense payment plan and optionally associated diagnostic and explanatory information.

The term “explanation text” refers to natural language text that describes or clarifies a diagnosis, a severity, or a medical expense payment plan in a form understandable to a user.

The term “server” refers to an information processing apparatus or a group of apparatuses that includes the processor and the information storage unit and that provides data processing functions and communication functions to the user terminal over a communication network.

In one embodiment, a server cooperates with a terminal operated by a user to implement the claimed system. The server includes at least one processor, a main memory, a non-volatile storage device, a communication interface, and an information storage unit implemented, for example, by a database system. The terminal includes at least one processor, a memory, a display, an input interface such as a touchscreen, and a wireless or wired communication module. The server and the terminal communicate over a communication network such as the Internet using a secure transport protocol.

The server executes an operating system such as a general-purpose server operating system, and application software implemented, for example, in a programming language such as Python. The server further executes a web framework such as a general-purpose application framework, a machine learning framework such as a neural network computation framework, and one or more libraries for natural language processing and numerical computation. The terminal executes a mobile operating system and an application that functions as a user interface client, which may be implemented as a native application or a web-based application communicating with the server.

The server stores, in the information storage unit, a prediction learning model configured as a neural network, one or more generative AI models, and medical expense payment plan data associated with severity levels of diseases. The prediction learning model is implemented on top of the neural network computation framework and may take the form of a multi-layer neural network, such as a feedforward network, a convolutional network, a recurrent network, or a hybrid architecture. In one specific example, the prediction learning model includes an input layer receiving numerical and categorical health-related features, several hidden layers each including multiple units with activation functions such as rectified linear unit functions, and an output layer producing probability scores for a plurality of disease classes and optionally a numerical severity score.

The server trains the prediction learning model in advance by using a training data set containing pairs of health condition information and ground-truth disease labels and severity annotations. The server defines a loss function such as a cross-entropy loss or a combination of cross-entropy loss and mean-squared error, and updates the weights of the neural network by a gradient descent-based optimization algorithm such as stochastic gradient descent, Adam, or a similar method. The server optionally applies regularization techniques such as dropout, weight decay, and early stopping to reduce overfitting. The server may also perform data augmentation for text data, for example by paraphrasing or synonym replacement, and for numerical data, for example by adding small perturbations within medically acceptable ranges, to improve generalization.

The server configures the generative AI models as transformer-based language models that process sequences of tokens representing prompt sentences and output sequences of tokens representing generated text. Each generative AI model includes an embedding layer, multiple transformer blocks each including a multi-head self-attention mechanism and a position-wise feedforward network, and a final output layer with a probability distribution over a vocabulary. The server trains or fine-tunes the generative AI models on corpora including medical texts, explanatory documents, and example dialogues so that the models can generate medically coherent and user-understandable analysis results and explanations. The server uses loss functions such as cross-entropy loss over token sequences and performs weight updates by gradient-based optimization. The server may store the trained parameters in the information storage unit and load them into memory at run time.

The server preprocesses health-related information received from the terminal into a structured data representation. The server converts numerical data such as body temperature and duration of symptoms into floating-point values, normalizes the numerical values according to predetermined ranges and statistics, and encodes categorical data such as chronic disease status as numerical vectors by one-hot encoding or embedding-based encoding. The server processes character data such as symptom descriptions by tokenizing the text into words or subword units, converting the tokens to indices according to a vocabulary, and optionally generating vector representations using word embeddings or contextual embeddings. By performing this structured preprocessing, the server reduces variability in input data, improves numerical stability of subsequent neural network computations, and enhances inference speed because the prediction learning model and the generative AI models can operate on compact numeric tensors rather than on arbitrary raw strings.

The server uses the preprocessed data to generate a first prompt sentence that will be input to an analysis generative AI model. The server constructs this prompt sentence by combining template segments and dynamic content. The server inserts normalized symptom descriptions, numerical measurements, and chronic disease indicators into a textual template that also includes explicit instructions for the generative AI model regarding the desired output format and reasoning depth. For example, the server generates a prompt sentence such as:

“The user's symptoms are headache and low-grade fever. Body temperature is 37.5 degrees. There are no chronic diseases. A preliminary machine learning model suggests a mild upper respiratory condition. As a medical assistant, list likely disease names, assign a severity level as mild, moderate, or severe, and provide a short explanation.”

The server thereby uses internal intermediate results from the prediction learning model to condition the prompt sentence, providing structured context that improves the quality, consistency, and stability of the output from the analysis generative AI model. This approach differs from conventional static prompts in that the server dynamically constructs the prompt according to specific numerical and categorical features, leading to more precise alignment between upstream numeric inference and downstream language-based reasoning.

The server inputs the first prompt sentence into the analysis generative AI model and receives as output an analysis result including at least one candidate disease name and a severity indication. The analysis generative AI model transforms the prompt sentence into token embeddings, applies multiple layers of self-attention to capture dependencies between symptom descriptions, numeric values, and preliminary diagnosis, and generates output tokens corresponding to disease names and severity labels. The server parses the generated text by using pattern matching rules and natural language processing techniques, thereby extracting structured elements such as disease identifiers, severity categories, and confidence phrases.

The server integrates the analysis result with a prediction result from the prediction learning model. The server obtains, from the prediction learning model, a vector of probabilities over disease classes and possibly a numerical severity score. The server defines integration rules or an additional integration module that combines the probabilistic output of the prediction learning model with the categorical and textual output of the analysis generative AI model. For example, the server may assign weights to the prediction learning model and the analysis generative AI model and compute a combined score for each disease candidate, or the server may use the prediction learning model's probabilities to filter or re-rank disease names proposed by the analysis generative AI model. The server then determines a final disease name and a final severity by selecting the candidate with the highest combined score subject to medically defined constraints.

By integrating the outputs in this manner, the server achieves a technical improvement in diagnostic accuracy and stability. The prediction learning model handles numerical and categorical patterns efficiently with high throughput, while the analysis generative AI model captures nuanced relationships in natural language descriptions. The integration reduces the impact of errors or biases in either model alone and provides a more robust and reliable final decision. This cooperative processing cannot be easily replicated by manual human reasoning at scale and provides an improvement to the functioning of the computer system in terms of error reduction and consistent handling of heterogeneous data.

The server maintains in the information storage unit a set of medical expense payment plan data. Each payment plan entry includes at least a severity level or range of severity levels, a total payment amount or formula, a number of installments, an interval between payments, and optional adjustment factors such as discounts or caps. The server associates each severity level or severity range with one or more candidate payment plans. The server uses the final severity determined by the integrated diagnostic process to retrieve one or more candidate plans from the information storage unit. The server may perform indexing or key-based lookup operations, and thus the retrieval process is computationally efficient even for large numbers of plan definitions.

The server generates payment plan presentation information based on the selected medical expense payment plan and the final diagnosis. To improve comprehensibility for the user, the server constructs a second prompt sentence for input to an explanation generative AI model. The server embeds in this second prompt sentence the final disease name, the severity, and the key parameters of the selected payment plan. For example, the server may generate a second prompt sentence such as:

“The final diagnosis is mild cold with mild severity. The recommended medical expense payment plan is a standard single payment of a specified amount. Generate a concise explanation to the user describing why this plan is suitable based on the mild severity and low risk.”

The server provides this second prompt sentence to the explanation generative AI model, which generates an explanation text. The server parses and optionally truncates or formats this explanation text and includes it in the payment plan presentation information together with the structured plan parameters.

The terminal receives the payment plan presentation information and displays it on a graphical user interface. The terminal shows, for example, the final disease name, the severity level, the payment amount, the number of installments if any, due dates, and the generated explanation text. The user reads these details and selects a preferred plan as allowed by the system. The terminal transmits the user's selection to the server, which records the selection in the information storage unit and may invoke further subsystems, such as billing or scheduling modules, to manage subsequent financial transactions. Although the business context involves payment planning, the technical contribution resides in the manner in which the server preprocesses data, generates prompt sentences, integrates multiple models, and structures payment plan data for efficient retrieval and presentation.

The server achieves several technical effects by the combination of the above-described components and operations. First, by converting heterogeneous health-related inputs into normalized numerical tensors and structured text tokens, the server reduces memory fragmentation and improves cache locality during neural network computation, which leads to improved processing speed. Second, by using a dedicated prediction learning model for pattern recognition on numeric and categorical features and a separate analysis generative AI model for language-based reasoning, and by integrating their outputs with explicit rules, the server reduces the computational load on each model while improving overall diagnostic accuracy. Third, by generating prompt sentences that encode structured intermediate results, the server improves the determinism and reproducibility of generative outputs, reducing the variability often associated with generative AI models and thereby reducing the need for repeated queries and network traffic, which in turn lowers communication load between the server and external AI services when such services are used.

In a further embodiment, the server implements the prediction learning model as a multi-task neural network that jointly predicts disease class probabilities and severity scores. The server defines a combined loss function with separate terms for disease classification and severity regression and uses a weighted sum of the terms to update network parameters. This architecture improves sample efficiency and ensures that the intermediate representations learned by the network capture both disease type and severity information in a unified latent space. In another embodiment, the server applies model quantization or pruning techniques to reduce the size of the neural network parameters and thereby reduce inference latency and memory usage, which enables deployment on resource-constrained servers.

In another embodiment, the server uses a rule-based layer in front of the analysis generative AI model to constrain prompt sentence generation. The server, for example, limits the vocabulary and phrase structures used for numerical ranges, severity labels, and plan types, and inserts explicit markers or delimiters into the prompt sentences. This rule-based formatting allows the server to parse the generated text more reliably and reduces post-processing complexity. This combination of rule-based formatting and neural inference represents a non-conventional and non-generic arrangement of components that improves system performance relative to traditional monolithic black-box models.

In a further embodiment, the server maintains a log of prompt sentences and corresponding outputs from the generative AI models in the information storage unit. The server analyzes the logs to detect patterns of model error or instability and adaptively adjusts prompt construction rules, such as changing phrasing or adding explicit constraints, to improve output quality. The server may also use these logs to fine-tune the generative AI models by additional training on successful prompt-output pairs. This feedback loop leads to progressive improvement of system behavior without requiring manual redesign of the entire processing pipeline.

The terminal may be implemented as a smartphone, a tablet, a personal computer, or a dedicated medical information terminal. The terminal can include local validation logic to prevent non-sensical values from being transmitted, such as temperatures outside a humanly feasible range. By performing some validation at the terminal, the system reduces unnecessary network traffic and server computation caused by invalid inputs, further improving system efficiency.

Across these embodiments, the server, the terminal, and the models cooperate in a technically specific way: the server performs structured preprocessing and integration steps, the generative AI models operate with carefully constructed prompt sentences, and the prediction learning model supplies numerically grounded disease estimates. This arrangement improves the functioning of the computer system beyond mere automation of human diagnostic and administrative tasks, by enhancing inference accuracy, stabilizing generative outputs, reducing resource consumption, and enabling modular extensibility of the system to new diseases, severity levels, and payment plan structures.

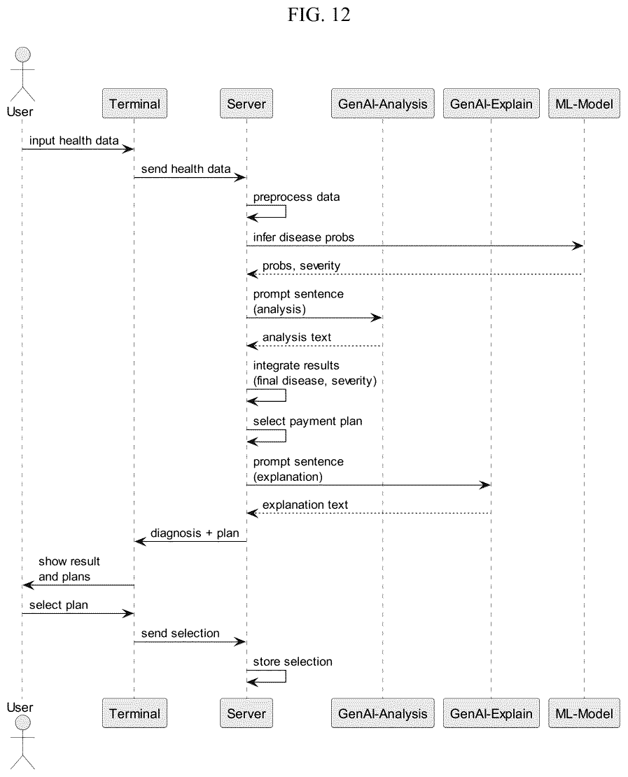

The following describes the processing flow using FIG. 12.

### Step 1:

The user operates the terminal to start a health support application and input health information. The user enters free-text symptom descriptions, numerical values such as body temperature, and categorical selections such as presence or absence of chronic diseases. The terminal receives these inputs as raw keystrokes and touch events and converts them into an internal data object containing text fields and numeric fields as input. The terminal outputs a structured request object including symptom text, numerical measurements, and categorical flags.

### Step 2:

The terminal validates the structured request object locally. The terminal checks that required fields are present, that numerical values lie within a plausible medical range, and that text fields do not exceed a defined maximum length. The terminal uses conditional checks and range comparisons on the input object. When invalid data is detected, the terminal outputs an error message to the user and requests correction; when the data passes validation, the terminal outputs a cleaned and validated request object ready for transmission to the server.

### Step 3:

The terminal sends the validated request object to the server over a communication network.

The terminal uses a communication module to encapsulate the request object into a network message, for example an HTTP request body encoded in a text-based or binary representation.

The input to this step is the cleaned and validated request object, and the output is a transmitted network packet containing the user's health information addressed to the server.

### Step 4:

The server receives the network packet via a communication interface and reconstructs the request object. The server parses the request body, converts it into an internal representation such as a dictionary or record structure, and verifies integrity by checking message headers and optional authentication tokens. The input to this step is the raw network packet, and the output is a server-side health information object containing symptom text, numerical data, and categorical attributes associated with a user identifier.

### Step 5:

The server performs preprocessing of numerical and categorical data. The server takes as input the numerical values such as body temperature and symptom duration and categorical values such as chronic disease flags. The server converts units if needed, normalizes numerical values using stored statistical parameters, and encodes categorical values into numerical vectors, for example by one-hot encoding. The server executes arithmetic operations, table lookups, and vector construction on the input data. The output is a set of standardized numerical feature vectors that are suitable for input to machine learning models.

### Step 6:

The server performs preprocessing of symptom text. The server takes as input the raw symptom text string contained in the health information object. The server applies tokenization, lowercasing, punctuation removal, and optional lemmatization, and then converts tokens into numerical indices according to a vocabulary. The server may further map token indices to embedding vectors using a stored embedding matrix. The server thereby performs string manipulation, dictionary lookups, and matrix indexing operations. The output is a text feature representation, such as a sequence of token indices or a tensor of embedding vectors.

### Step 7:

The server generates a first prompt sentence for an analysis generative AI model. The server uses as input the standardized numerical feature vectors, the text feature representation, and optionally an intermediate prediction from a separate prediction learning model. The server inserts values such as symptom phrases, normalized temperatures, and chronic disease statuses into a predefined textual template and appends explicit instructions for desired output structure and severity classification. The server concatenates strings and converts numeric values into human-readable text. The output is a first prompt sentence in natural language that encodes both raw and processed health information in a form suitable for the analysis generative AI model.

### Step 8:

The server invokes the analysis generative AI model by supplying the first prompt sentence. The server converts the prompt sentence into tokens, passes the token sequence into the generative AI model, and receives model-generated text as output. Inside this step, the generative AI model performs internal numerical computations such as embedding lookup, self-attention, and matrix multiplications to produce output tokens. The input to this step is the first prompt sentence, and the output is an analysis text that includes one or more candidate disease names and corresponding severity descriptions.

### Step 9:

The server parses and structures the analysis text. The server uses pattern matching, rule-based extraction, or light natural language processing to identify disease names, severity labels, and optional explanatory phrases within the analysis text. The server applies string search, regular expressions, and dictionary mapping from text labels to internal identifiers. The input is the free-form analysis text, and the output is a structured analysis result object containing a set of candidate diseases, each with an associated severity level and optional explanatory metadata.

### Step 10:

The server obtains a prediction result from a prediction learning model. The server uses as input the standardized numerical feature vectors and the text feature representation produced in previous preprocessing steps. The server formats these features into an input tensor and feeds the tensor into the prediction learning model, which performs neural network inference operations such as weighted sums, activation functions, and softmax normalization. The output of this step is a prediction result that includes probability scores over disease classes and, optionally, a continuous or discrete severity score.

### Step 11:

The server integrates the structured analysis result with the prediction result. The server aligns disease names from the analysis result with disease classes from the prediction result by using mapping tables or similarity matching. The server computes combined scores for each disease candidate using a predefined integration rule, for example a weighted sum or a rule-based selection that prioritizes agreement between models. The server compares combined scores and applies thresholding to select a final disease name and a final severity. The input to this step is the analysis result object and the prediction result, and the output is a final diagnosis object containing a single disease name and a corresponding severity category or numerical level.

### Step 12:

The server retrieves medical expense payment plan data corresponding to the final severity. The server uses as input the final severity value and accesses an information storage unit containing multiple payment plan records indexed by severity or severity ranges. The server performs a database query or key-based lookup, filters candidate plans according to additional criteria such as user eligibility or policy rules, and selects at least one applicable payment plan. The output is a selected payment plan object including parameters such as total cost, number of installments, installment amount, and payment schedule.

### Step 13:

The server generates a second prompt sentence for an explanation generative AI model. The server takes as input the final diagnosis object and the selected payment plan object. The server constructs a textual description containing the final disease name, the severity, and the key payment plan parameters, and then embeds this description in a template that instructs the generative AI model to produce a user-friendly explanation. The server performs string concatenation, numeric-to-text conversions, and insertion of explanatory phrases. The output is a second prompt sentence that instructs the explanation generative AI model to generate an explanation text for the recommended medical expense payment plan.

### Step 14:

The server invokes the explanation generative AI model using the second prompt sentence. The server tokenizes the second prompt sentence and forwards the tokens to the explanation generative AI model, which performs transformer-based computations and generates output tokens. The server receives the generated tokens and converts them back into a text string.

The input to this step is the second prompt sentence, and the output is an explanation text that describes the relationship between the diagnosis, the severity, and the recommended payment plan in natural language.

### Step 15:

The server assembles payment plan presentation information. The server combines the final diagnosis object, the selected payment plan object, and the generated explanation text into a single response structure. The server formats numerical values, such as installment amounts and dates, for display and attaches identifiers necessary for tracking and auditing. The server thereby transforms multiple internal objects into a presentation data object configured for direct consumption by the terminal. The input to this step is the final diagnosis, payment plan, and explanation text, and the output is a payment plan presentation object.

### Step 16:

The server transmits the payment plan presentation object to the terminal. The server encodes the presentation object into a message, attaches necessary metadata such as user identifiers and timestamps, and sends the message over the communication network using a communication protocol. The input is the payment plan presentation object, and the output is a network packet delivered to the terminal that carries all information required for user display.

### Step 17:

The terminal receives the network packet and prepares display data. The terminal parses the received message, reconstructs the payment plan presentation object, and maps its fields to visual components such as text labels, tables, and buttons. The terminal may convert internal identifiers into human-readable labels using local resources. The input to this step is the received network packet, and the output is a set of display parameters and UI elements ready for rendering on the terminal screen.

### Step 18:

The user views the displayed diagnosis and payment plan information on the terminal and selects a preferred option. The user reads the disease name, the severity, the payment amounts, and the explanation text, and then interacts with UI elements such as buttons or selection lists to choose one payment plan. The input to this step is the rendered screen showing available plans, and the output is a selection action captured as a user interaction event on the terminal.

### Step 19:

The terminal converts the user interaction event into a confirmation message. The terminal records the selected plan identifier, associates it with the ongoing session and user identifier, and constructs a confirmation object. The terminal may perform a final local validation, such as ensuring that a required consent checkbox is checked. The input to this step is the user interaction event, and the output is a confirmation message object indicating the chosen payment plan.

### Step 20:

The terminal sends the confirmation message to the server. The terminal packages the confirmation object into a network message, attaches authentication data, and transmits the message to a designated endpoint on the server. The input to this step is the confirmation message object, and the output is a network packet containing the user's confirmed selection.

### Step 21:

The server receives the confirmation packet and updates stored records. The server parses the confirmation message, identifies the related diagnosis session and user, and writes the selected plan and status into the information storage unit. The server uses database insert or update operations to persist the selection and may trigger additional modules such as billing or scheduling systems. The input is the confirmation message, and the output is an updated data record representing the finalized association between the user, the diagnosis, and the payment plan.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

Conventional computer-implemented medical support systems typically treat different input modalities, such as medical images and text-based interview information, in a fragmented and sequential manner. In many systems, image analysis is performed by a dedicated image recognition engine, while symptom descriptions and interview answers are processed by a separate text engine or by simple rule-based logic. These subsystems often produce independent outputs that are loosely combined at a later stage by fixed heuristics. As a result, the system cannot fully exploit correlations between visual features and textual symptom contexts, leading to sub-optimal diagnostic suggestions and unreliable assessment of severity and consultation urgency.

In addition, conventional systems generally rely on static classification models that are trained to output only a limited set of disease labels or risk scores. Such models are not designed to flexibly generate patient-oriented explanations, structured reasoning, or tailored recommendations for medical facilities and pharmaceuticals. When these explanations or recommendations are later added by separate rule-based modules or human operators, the processing pipeline becomes complex, difficult to maintain, and prone to inconsistency between the underlying analysis and the displayed guidance.

Further, in many existing architectures, the prompt construction for a generative artificial intelligence model, if used at all, is handled in an ad hoc manner outside the core processing flow. The image analysis results and structured symptom data are not systematically integrated into a machine-readable case representation that can be robustly transformed into a prompt sentence. Consequently, the generative model may receive incomplete or poorly contextualized input, which reduces the accuracy, consistency, and repeatability of generated diagnostic support information.

Moreover, conventional systems often do not provide a unified computer-implemented mechanism to connect diagnostic analysis with downstream actions, such as selecting an appropriate medical service provider based on patient condition, location, and facility attributes, or identifying candidate pharmaceuticals and articulating usage precautions. These downstream tasks are typically performed manually or with separate applications, increasing latency and limiting the scalability of the overall solution.

From a computer technology perspective, there is therefore a need for an improved server-side architecture and processing method that: (i) securely receives and preprocesses multimodal patient input data; (ii) performs integrated feature extraction and structuring of both image and text data; (iii) automatically constructs rich, context-sensitive prompt sentences for a generative artificial intelligence model; (iv) uses the generative artificial intelligence model not only for listing diagnostic candidates but also for generating explanations, severity-aware guidance, and recommendations; and (v) systematically links model outputs with machine-readable severity information, medical facility attributes, and pharmaceutical data. Such an architecture should improve the technical functioning of the medical support system by reducing manual configuration, improving data integration, and enabling more accurate, consistent, and scalable generation of diagnostic support information.

The specific processing by the specific processing unit 290 of the data processing device 12 in Example 2 is realized by the following means.

The present invention provides a server comprising a processor and a storage device, the processor being configured to execute computer-implemented processing to receive image data and text data acquired from an information processing terminal operated by a user via a secure communication protocol, preprocess the received image data by performing at least standardization and noise reduction to generate analysis data, and store the preprocessed image data and the text data in the storage device; input the preprocessed image data into an image recognition artificial intelligence model based on convolution operations, extract feature data from the image data, and generate candidate disease information by comparing the feature data with record information stored in the storage device and including symptom information on the basis of a similarity measure; perform natural language processing on the text data to detect medical entities, and convert the text data into structured data including at least symptom content, onset location, elapsed time, accompanying symptoms, and medical history information; integrate the candidate disease information and the structured data, generate case information summarizing an image analysis result and interview information, and automatically construct a prompt sentence configured to cause a generative artificial intelligence model to generate diagnosis candidates and explanatory information on the basis of the case information; input the prompt sentence to the generative artificial intelligence model and obtain diagnostic support information including at least candidate disease names, reasoning content, and a consultation urgency level; access severity information stored in the storage device and associated with the candidate disease names to generate evaluation information including a severity evaluation result; and convert the diagnostic support information and the evaluation information into display information in a format transmittable to the information processing terminal and transmit the display information to the information processing terminal. This enables an integrated, computer-implemented pipeline that improves the technical functioning of the medical support system by securely acquiring multimodal patient data, performing coordinated image and text analysis, generating structured case information, automatically forming context-rich prompt sentences for a generative artificial intelligence model, and producing consistent, severity-aware diagnostic support outputs and recommendations in a form directly usable by client devices.

The term “system” refers to an arrangement of one or more hardware devices and software components that cooperate to execute the processing described herein.

The term “server” refers to an electronic computing apparatus, typically including one or more processors, a memory, a storage device, and communication interfaces, that executes programs to provide services over a communication network.

The term “processor” refers to one or more hardware circuits, such as a central processing unit or a graphics processing unit, configured to execute instructions to perform arithmetic, logic, control, and input/output operations.

The term “storage device” refers to any non-transitory computer-readable medium, such as a semiconductor memory, magnetic storage, or optical storage, that stores data and programs used by the processor.

The term “information processing terminal” refers to an electronic device operated by a user, such as a portable communication device, a tablet device, or a general-purpose computer, that can capture data, execute applications, and communicate with the server.

The term “user” refers to a human operator, such as a patient or caregiver, who interacts with the information processing terminal to provide data and receive information.

The term “image data” refers to digital data representing a still image or sequence of images, including at least pixel values that depict a physical object such as a body part or skin region.

The term “text data” refers to character data representing natural language input, including words, sentences, or phrases describing symptoms, medical history, or other information.

The term “secure communication protocol” refers to a communication procedure that provides at least encryption and integrity protection for data transmitted between the information processing terminal and the server.

The term “preprocessing” refers to a set of operations applied to raw input data to convert the data into a format suitable for analysis, including at least standardization, normalization, and noise reduction.

The term “standardization” refers to adjusting one or more attributes of data, such as size, resolution, color space, or numerical range, to conform to predefined conditions used by a model or algorithm.

The term “noise reduction” refers to processing that suppresses undesired variations or artifacts in data, such as random pixel fluctuations in image data, while preserving relevant features.

The term “analysis data” refers to data obtained after preprocessing, which is formatted and conditioned for input into an analysis model or algorithm.

The term “image recognition artificial intelligence model” refers to a computational model, implemented by machine learning or deep learning techniques, that analyzes image data to extract features and infer properties such as object categories or patterns.

The term “convolution operations” refers to mathematical operations in which a kernel is applied across input data, such as an image, to compute local feature responses used in neural network layers.

The term “feature data” refers to numeric values, such as vectors or tensors, that represent characteristics extracted from input data, including patterns related to color, texture, shape, or structure.

The term “candidate disease information” refers to information indicating one or more possible disease conditions, including identifiers, labels, or probabilities associated with each candidate.

The term “record information” refers to data stored in the storage device, including symptom information, case information, and associated metadata used for comparison or retrieval.

The term “symptom information” refers to data describing physical or subjective signs of a health condition, such as rash, pain, swelling, fever, or duration.

The term “similarity measure” refers to a quantitative value representing likeness between two sets of data, such as feature vectors, computed by functions including cosine similarity or distance metrics.

The term “natural language processing” refers to computational techniques for analyzing and interpreting human language text to identify linguistic structures and semantic entities.

The term “medical entity” refers to a concept related to healthcare, such as a symptom, body location, time expression, disease name, medical history element, or medication.

The term “structured data” refers to data organized according to a defined schema, such as key-value pairs or database fields, that can be programmatically accessed and processed.

The term “symptom content” refers to a portion of structured data that specifies the type or description of a symptom reported by the user.

The term “onset location” refers to a portion of structured data that indicates the body part or region where a symptom appears.

The term “elapsed time” refers to a portion of structured data that represents a temporal duration or period from symptom onset to the time of reporting.

The term “accompanying symptoms” refers to symptoms that occur together with a main symptom, such as itching with a rash or fever with pain.

The term “medical history information” refers to data regarding past diseases, treatments, surgeries, allergies, or other health-related events of the user.

The term “integrate” refers to combining multiple types of data or information, such as candidate disease information and structured data, into a single composite representation.

The term “case information” refers to aggregated data describing a particular user's situation, including image analysis results, structured symptom data, and any associated metadata.

The term “image analysis result” refers to output produced by analyzing image data, including extracted feature data, identified patterns, and related candidate disease information.

The term “interview information” refers to information obtained from user responses to questions about symptoms, lifestyle, or medical history, represented as text data or structured data.

The term “prompt sentence” refers to a text input that instructs a generative artificial intelligence model to perform a specific task, such as generating diagnosis candidates and explanations, based on included context.

The term “generative artificial intelligence model” refers to an artificial intelligence model configured to generate new content, such as natural language text, in response to input data and prompt sentences.

The term “diagnosis candidates” refers to one or more possible diseases or health conditions suggested by analysis of user data, without constituting a definitive medical diagnosis.

The term “explanatory information” refers to text generated to clarify why certain diagnosis candidates are suggested, including reasoning based on features and symptoms.

The term “diagnostic support information” refers to information generated to assist in medical decision-making, including diagnosis candidates, explanatory information, and consultation guidance.

The term “candidate disease names” refers to textual identifiers of diseases or conditions that are suggested as possible explanations of the user's symptoms.

The term “reasoning content” refers to information describing logical or probabilistic grounds for associating specific candidate disease names with observed data.

The term “consultation urgency level” refers to an indication of how quickly a user should seek professional medical attention, such as immediate, urgent, or routine.

The term “severity information” refers to data indicating the seriousness or risk level associated with specific diseases or conditions.

The term “evaluation information” refers to data generated by assessing severity information in relation to candidate disease names, including severity evaluation results.

The term “severity evaluation result” refers to a determination, based on severity information and candidate diseases, of an overall severity ranking or classification for a case.

The term “display information” refers to data formatted for presentation on an information processing terminal, including text, structured fields, and associated indicators.

The term “position information” refers to data representing a geographic location related to the user or device, such as coordinates or region identifiers.

The term “attribute information of medical service providers” refers to data describing characteristics of medical facilities, including departments, available equipment, operating hours, and capacity.

The term “medical service provider” refers to an entity that delivers healthcare services, such as a hospital, clinic, or pharmacy.

The term “medical department” refers to a classification of medical specialty, such as dermatology, internal medicine, or pediatrics.

The term “available facilities” refers to resources or equipment at a medical service provider, such as diagnostic devices, treatment capabilities, or emergency services.

The term “consultation priority” refers to a relative ranking that indicates how strongly a specific medical service provider should be recommended for a particular case.

The term “recommendation message” refers to information generated to suggest one or more medical service providers and to state reasons or conditions for such suggestions.

The term “pharmaceutical information” refers to data concerning medicinal products, including active ingredients, indications, contraindications, dosage, and usage precautions.

The term “candidate pharmaceutical information” refers to pharmaceutical information filtered or selected as potentially suitable based on candidate disease information and structured data.

The term “guidance information” refers to information generated to instruct or advise a user regarding suitable pharmaceuticals and their use, including precautions and usage conditions.

The term “input conditions” refers to a set of data items provided to a generative artificial intelligence model through a prompt sentence, which define the context and constraints for generation.

In one embodiment, a server, a terminal, and a user cooperate to implement a medical diagnostic support system that integrates image analysis, text analysis, and generative artificial intelligence processing. The server includes at least one processor, a main memory, a non-transitory storage device, and one or more network interfaces. The terminal includes a processor, a display device, a camera, a user input interface, and a communication module.

The user operates the terminal to capture and submit information about a health condition, and the server executes a sequence of computer-implemented operations to generate diagnostic support information and recommendations.

The terminal acquires image data using an integrated camera. The terminal uses operating system camera APIs (for example, a camera framework of a mobile operating system) to capture a digital image of a physical region of the user's body, such as skin. The terminal converts the raw sensor output into an image file in a standard format, such as JPEG or PNG, by invoking an imaging library that performs demosaicing, color correction, and compression.

The terminal optionally performs local resizing or orientation correction to reduce file size and normalize orientation before transmission, thereby reducing network load and improving end-to-end processing latency.

The terminal acquires text data from the user through graphical user interface components.

The user inputs free-form symptom descriptions and answers to structured questions, such as “When did the symptom start?”, “Where on your body is the symptom located?”, and “Do you have fever?”. The terminal stores the user's responses as character strings and as simple key-value pairs (for example, keys such as “symptom_description” or “duration_input”). The terminal then packages the image data and text data into one or more request messages, typically represented as a structured network payload (for example, a multipart HTTP request containing binary image data and a text body). The terminal establishes a secure transport channel using a secure communication protocol, such as a protocol providing encryption and integrity verification, and transmits the payload to the server via the network interface.

The server receives the incoming request using a network interface controlled by a communication stack. The server validates the integrity and format of the received message and stores the image data and the text data in a storage device. The storage device holds at least an image repository, a case database, a symptom database, a severity database, a facility database, and a pharmaceutical database. The server associates each received image and corresponding text data with a unique case identifier and records metadata such as timestamp, terminal type, and approximate user location (if provided by the terminal).

The server preprocesses the image data using an image processing library, such as an implementation of image manipulation routines that support resizing, color conversion, and noise filtering. The server converts the image into a fixed resolution and color space required by an image recognition artificial intelligence model. For example, the server converts the image into a three-channel tensor of fixed width and height (e.g., 224 by 224 pixels) and normalizes pixel values to a predefined numeric range (e.g., floating-point values between 0 and 1). The server optionally applies noise reduction filters, such as a Gaussian filter or a median filter, to reduce sensor noise and compression artifacts while preserving relevant lesion edges. By applying these controlled transformations, the server ensures that the subsequent model receives data in a consistent format, which improves numerical stability and recognition accuracy.

The server executes an image recognition artificial intelligence model based on a convolutional neural network architecture. In one embodiment, the server uses a deep neural network comprising an input layer, multiple convolutional layers, non-linear activation layers, pooling layers, and one or more fully connected layers. The server stores model parameters, including learned weights and biases, in the storage device and loads them into main memory at runtime. The server may deploy the model using a general-purpose deep learning framework that executes tensor operations on a central processing unit or a graphics processing unit.

The server feeds the preprocessed image tensor into the convolutional neural network. Each convolutional layer applies multiple learned kernels over the input feature maps, computing dot products and producing intermediate activation maps. Non-linear activation functions, such as rectified linear units, introduce non-linearity, while pooling layers, such as max pooling or average pooling, reduce the spatial dimension and provide translational invariance. The server propagates the data through the network layers until a lower-dimensional feature representation is obtained in one or more penultimate layers. The server treats this representation as feature data, for example, as a high-dimensional vector encoding color distribution, lesion shape, border sharpness, and texture patterns. The server then optionally applies a classification head that outputs a probability distribution over predefined disease categories.

The server compares the feature data to existing record information stored in a symptom database. The symptom database contains, for each reference case, one or more feature vectors, disease labels, and associated symptom metadata. The server computes one or more similarity measures, such as cosine similarity or Euclidean distance, between the feature data of the current case and the stored feature data of multiple reference cases. The server selects a subset of reference cases that exhibit the highest similarity to the current case and aggregates corresponding disease labels and similarity scores to form candidate disease information. This operation leverages the structure of the learned feature space to efficiently search for similar medical images, which improves diagnostic candidate recall and reduces false positives compared to simpler rule-based image filters.

The server preprocesses the text data by cleaning and normalizing the input. The server removes control characters, normalizes whitespace, and standardizes typical medical units and expressions (for example, converting various textual forms of temperature into a common unit representation). The server logs the raw and normalized text to the storage device for auditing and model improvement, with appropriate anonymization or pseudonymization.

The server performs natural language processing on the text data using a text analysis pipeline. In one embodiment, the server uses a language model that includes tokenization, part-of-speech tagging, and named entity recognition components. The server segments the text into tokens, assigns syntactic categories, and identifies medical entities such as body locations (“left forearm”), symptoms (“red rash”, “itchy”), temporal expressions (“2 days”), and negations (“no fever”). The server then maps the recognized entities into a structured schema. For example, the server constructs a structured data object that includes fields such as “main_symptom”, “additional_symptom”, “location”, “duration”, and “fever_presence”. The server stores this structured data in the case database for subsequent integration with image-based information.

The server integrates the candidate disease information and the structured data by constructing case information. The server generates a combined representation that includes image analysis results (feature data, candidate diseases, and similarity scores) and structured symptom data (locations, durations, and other entities). The server may apply rule-based weighting or statistical adjustment to refine candidate rankings, for example, reducing the score of diseases that are inconsistent with reported absence of fever or adjusting for body location. This integration step uses explicit rules and weightings that are applied by the server's processor and are not ordinarily performed by human operators manually, thereby improving consistency and repeatability of diagnostic support.

The server constructs a prompt sentence for a generative AI model based on the case information. The server uses a template that includes sections for instructions, image findings, candidate conditions, questionnaire summary, and output requirements. The server programmatically fills in the template with values from the case information. An example of such a prompt sentence is:
“You are a medical assistant AI. A patient has submitted a skin image and answered a questionnaire.

Image analysis (CNN) suggests:
Possible conditions: allergic contact dermatitis (0.62), urticaria (0.25), eczema (0.10).
Image features: red maculopapular rash localized to the left forearm, irregular borders, no visible bleeding.

Questionnaire (converted to structured data):
Location: left forearm
Main symptom: red rash
Additional symptom: itching
Duration: 2 days
Fever: none
New medications or cosmetics: none reported

Based on this information, use your medical reasoning to:
1. List the most likely disease names in order of likelihood.
2. Explain briefly, in lay terms, why you think each condition is possible.
3. Indicate whether the patient should seek urgent care, non-urgent in-person consultation, or self-care with monitoring.

Respond in concise English suitable for a patient.”

The server transmits this prompt sentence to the generative AI model, together with configuration parameters such as maximum output length and diversity settings. The generative AI model may be a large language model or a multimodal model trained on large corpora of text and potentially image-text pairs, implemented as a neural network with an encoder-decoder or transformer architecture. The server does not treat the generative AI model as a black box; instead, the server configures tokenization, attention mechanisms, and decoding strategies (such as beam search or nucleus sampling) through explicit parameters in the model interface, which directly influence computational behavior and output determinism.

The server obtains a textual output from the generative AI model that includes diagnosis candidates, reasoning content, and a consultation urgency level. The server optionally requires the model to output in a semi-structured format, such as enumerated items or labeled sections, to facilitate downstream parsing. The server parses the output to extract individual candidate disease names, explanations, and urgency indicators. The server records this diagnostic support information in the case database.

The server evaluates severity based on candidate disease names by querying a severity database. The severity database holds, for each disease or condition, structured severity information such as typical risk level, warning signs, and recommended consultation windows. The server retrieves the severity characteristics corresponding to each candidate disease name and computes a severity evaluation result for the case. The server may apply a weighting algorithm that considers both the generative AI model's ranking and the stored severity levels to produce an aggregate severity classification, such as “high”, “medium”, or “low”.

The server generates display information by combining the diagnostic support information and the evaluation information into a format suitable for rendering on the terminal. The server structures the display information into sections, including a summary of likely conditions, explanation in plain language, severity flags, and consultation recommendations. The server compresses or encodes the display information as needed and transmits it back to the terminal via the secure communication protocol.

The terminal receives the display information, decodes it, and presents it to the user on the display device. The terminal may highlight urgent messages using a distinctive color or icon and may present buttons that allow the user to access further details or initiate contact with a medical service provider. The user can review the suggested diagnoses, read the explanations, and decide whether to seek professional medical attention based on the generated guidance.

In one embodiment corresponding to a secondary claim, the server uses the case information and evaluation information to select an appropriate medical service provider. The server accesses a facility database that contains, for each medical service provider, attribute information such as medical departments, available equipment, opening hours, and geographic coordinates. The server computes a match score between the user's case and each facility based on factors including required specialization, severity, and proximity. The server then constructs a prompt sentence for the generative AI model that describes the selected facilities and the case context and requests generation of a recommendation message explaining why particular facilities are suitable. The server returns the resulting recommendation message to the terminal as part of the display information, providing the user with an actionable, technically grounded suggestion for where to seek care.

In another embodiment, the server accesses a pharmaceutical database that contains pharmaceutical information including active ingredients, indications, contraindications, dosage ranges, and precautions. The server filters this database based on candidate disease information and structured symptom data to extract candidate pharmaceutical information.

The server then constructs a prompt sentence that includes disease candidates, symptom details, and the filtered candidate pharmaceuticals, and requests, from the generative AI model, guidance information including suitable pharmaceutical options and usage precautions. The server reviews and, if desired, constrains the content (for example, to ensure that recommendations remain informational rather than prescriptive) and sends the guidance information to the terminal for display to the user.

From a technical perspective, the described architecture improves computer functionality in several ways. The server uses consistent data structures-such as fixed-size image tensors, high-dimensional feature vectors, and schema-based structured symptom objects—to reduce complexity in downstream processing. By enforcing these representations, the server reduces parsing overhead and avoids ambiguous data interpretations that could otherwise require manual intervention. The integration of image-based feature vectors with structured text-derived fields into a unified case information object enables the server to perform more efficient query operations on databases, thereby reducing computational cost for similarity search and candidate ranking.

The server also improves processing speed and accuracy by combining specialized models. The convolutional neural network is optimized for extracting discriminative visual features from images, while the natural language processing pipeline is optimized for capturing semantic content from text. The server integrates outputs from these models using explicit algorithms, such as similarity-based matching and rule-based weighting, which are tuned for computational efficiency and can be executed in parallel on multi-core processors or specialized accelerators. This modular yet tightly coupled architecture reduces the latency between receiving raw data and producing diagnostic support information compared with sequential, loosely coupled systems.

The server's approach to prompt generation further improves computational efficiency and output quality. Rather than allowing arbitrary, manually created prompts, the server uses programmatic templates and deterministic insertion of structured data to form context-rich prompt sentences. This reduces variability in the input to the generative AI model, stabilizes its internal attention patterns, and leads to more consistent outputs for similar cases. Because the prompt content is automatically derived from the structured case representation, the system reduces operator burden and avoids human errors in describing the case to the model. This design also supports reproducibility and explainability, since the relationship between input data, intermediate representations, and generated text can be traced and audited.

The learning processes used to construct the convolutional neural network and the generative AI model include detailed algorithmic techniques. During model training, a training subsystem (which may be implemented on the server or on a separate training platform) uses large sets of labeled images and associated diagnostic labels to optimize network weights. The training algorithm splits the data into training and validation subsets, applies data augmentation operations such as random rotations, scaling, flips, and color jitter, and feeds augmented inputs into the network. A loss function, such as cross-entropy loss for classification, measures the discrepancy between predicted outputs and ground truth labels. An optimization algorithm, such as stochastic gradient descent or an adaptive gradient method, computes parameter updates by backpropagating gradients and adjusts the weights iteratively until convergence criteria are satisfied. These learning procedures produce a neural network with high discriminative power and robustness to variations in image acquisition conditions.

Similarly, the generative AI model is trained using large corpora of text, optionally integrated with structured medical texts and user queries. The training procedure uses a language modeling objective, such as predicting the next token given prior tokens, and may incorporate supervised fine-tuning using example prompt-completion pairs aligned to medical explanation tasks. The training system computes a loss function that penalizes mismatches between predicted and target token sequences and performs gradient-based weight updates. Such structured training improves the model's ability to generate coherent, context-sensitive explanations when provided with the prompt sentences constructed by the server.

The described system does not merely automate a human mental process. The server performs complex, high-dimensional numerical operations, similarity computations in an embedded feature space, deterministic structuring of natural language into machine-interpretable fields, and template-driven prompt generation that is specifically designed for machine consumption. The system uses these technical mechanisms to manage bandwidth (by normalizing and compressing images), to optimize storage access patterns (by indexing feature vectors and structured records), and to reduce computational redundancy (by reusing extracted features in multiple downstream tasks). As a result, the technical effects include improved accuracy of diagnostic candidate generation, reduced end-to-end processing time, improved utilization of storage and communication resources, and increased reliability and consistency of outputs compared with conventional systems that rely on loosely coupled modules or purely rule-based logic.

Various modifications and alternative embodiments can be adopted without departing from the technical scope supported by the claims. For example, the server may replace or supplement the convolutional neural network with a vision transformer or hybrid architecture if such models provide improved feature representations. The natural language processing pipeline may employ different tokenization strategies or may incorporate domain-specific language models. The similarity search over feature vectors may be implemented using approximate nearest neighbor algorithms in high-dimensional indexing structures to further accelerate retrieval. The generative AI model may be hosted by the same server, by a separate inference server, or by a cloud-based model provider, as long as the server maintains control over prompt construction and output post-processing. The facility and pharmaceutical databases may be maintained in different physical storage systems or may be integrated into a single logical database. All such variations can be realized using standard computing hardware and software by following the system architecture and data processing mechanisms described above.

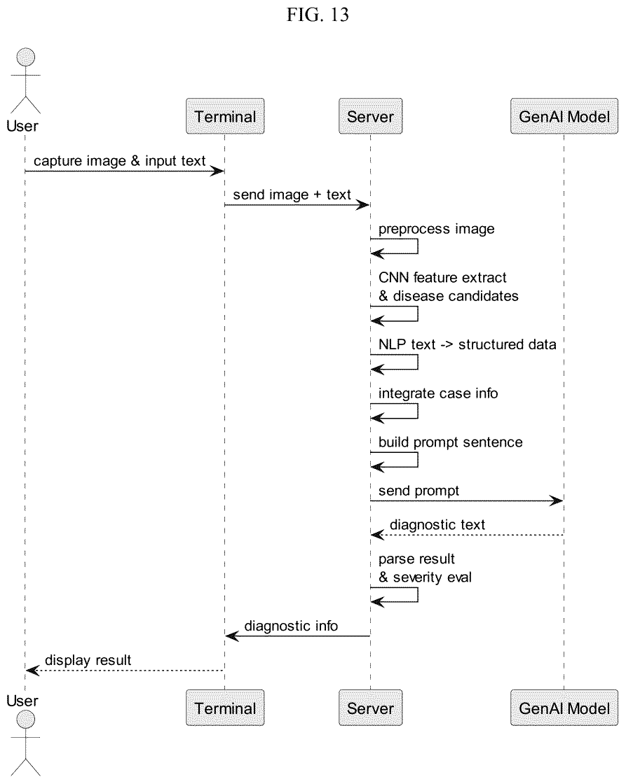

The following describes the processing flow using FIG. 13.

### Step 1:

User operates the terminal to capture symptom images and input symptom text.

User uses the camera function of the terminal to photograph a physical region, such as skin with a rash. The input to this step is the real-world scene of the user's body and the user's intent to record symptoms. The terminal converts light captured by the camera sensor into raw pixel data and then into a compressed image file (for example, JPEG or PNG) using an imaging library. User also enters symptom descriptions and answers to questions (for example, “Red rash on left forearm, itchy, started 2 days ago, no fever.”) via text fields, buttons, or checkboxes. The output of this step is digital image data and text data stored temporarily in the terminal's memory.

### Step 2:

Terminal packages and securely transmits image data and text data to the server. Terminal takes the captured image file and the collected text strings as input. Terminal optionally resizes the image, adjusts orientation, and compresses the file to reduce size.

Terminal constructs a network payload (for example, an HTTP POST request with multipart form data) that includes the binary image and a structured body containing text fields.

Terminal establishes a secure connection using a secure communication protocol and sends the payload to the server through its communication module. The output of this step is a network message containing image data and text data delivered to the server.

### Step 3:

Server receives the request, validates it, and stores raw data.

Server uses a network interface and a web application framework to accept the incoming request as input. Server checks headers, verifies that required parts such as an image and symptom text are included, and rejects malformed or incomplete requests. Server then writes the image file to an image repository in a storage device and inserts a new record in a case database that includes a case identifier, a pointer to the image file, the raw text data, timestamps, and optional location metadata. The output of this step is a stored case record that links the raw image data and raw text data under a unique case ID.

### Step 4:

Server preprocesses the image data into a normalized image tensor.

Server loads the stored image file for the given case ID as input using an image processing library. Server converts the image to a predetermined color space (for example, RGB), resizes it to a fixed resolution required by the model (for example, 224×224 pixels), and applies noise reduction filters (for example, Gaussian blur) if configured. Server scales pixel values from integer [0, 255] to floating-point values in a normalized range and arranges the values into a multidimensional array with a defined shape (for example, [channels, height, width]).

Through these operations, server performs data transformations that remove variability in size and intensity while preserving lesion features. The output of this step is a normalized image tensor ready for input to the image recognition model.

### Step 5:

Server executes a convolutional neural network to extract image feature data and preliminary disease scores.

Server takes the normalized image tensor as input and feeds it into a convolutional neural network implemented in a deep learning framework. Server performs a sequence of tensor operations: convolutions, bias additions, non-linear activations, and pooling. Each convolution layer aggregates local pixel neighborhoods into higher-level visual patterns, while pooling layers reduce spatial resolution and improve translational invariance. Server processes the activations through multiple layers to compute a final feature vector and, optionally, a probability distribution over predefined disease labels using fully connected layers and a softmax function. The output of this step is high-dimensional feature data representing the image and, when present, preliminary disease probability scores.

### Step 6:

Server matches the extracted feature data with reference cases to generate candidate disease information.

Server uses the feature vector from Step 5 and retrieves a collection of stored feature vectors and labels from a symptom database as input. Server computes similarity metrics such as cosine similarity between the current feature vector and each stored vector or an indexed subset. Server ranks reference cases by similarity and aggregates their associated disease labels and similarity scores into a list. Server may combine these similarity scores with the preliminary disease probabilities from the model to refine ranking. The output of this step is candidate disease information that includes candidate disease names and associated confidence or similarity values.

### Step 7:

Server preprocesses and normalizes the text data.

Server reads the raw symptom text associated with the case from the case database as input. Server removes control characters and extra whitespace, standardizes punctuation, and normalizes units and common expressions (for example, converts “38.5C” and “38.5° C.” into a consistent representation such as “38.5° C.”). Server logs both raw and normalized text for later analysis and improvement. The output of this step is cleaned, normalized text data suitable for natural language processing.

### Step 8:

Server performs natural language processing to create structured symptom data.

Server feeds the normalized text as input to a text analysis pipeline. Server tokenizes the text into words or subword units, assigns part-of-speech tags, and uses named entity recognition to detect medical entities such as symptoms (“red rash”), body locations (“left forearm”), durations (“2 days”), and negations (“no fever”). Server then maps these entities to fields in a predefined schema. For example, server may set “main_symptom=red rash”, “additional_symptom=itching”, “location=left forearm”, “duration_days=2”, and “fever=false.” Server stores this structured data object in the case database. The output of this step is structured symptom data that represents the text input in a machine-readable, schema-based format.

### Step 9:

Server integrates image-based candidate disease information and structured symptom data into case information.

Server takes as input the candidate disease information from Step 6 and the structured symptom data from Step 8. Server combines these into a single case information object, adding metadata such as feature vector identifiers, similarity scores, and any rule-based adjustments. Server may apply explicit rules such as down-weighting conditions that usually involve fever when the “fever” field is false or filtering out conditions that rarely occur at the specified body location. Server computes updated candidate scores according to these rules and stores the combined result in the case database. The output of this step is unified case information that contains both visual and textual evidence for use in subsequent reasoning.

### Step 10:

Server constructs a context-rich prompt sentence for a generative AI model.

Server uses the unified case information as input, including image features, candidate conditions, and structured symptom fields. Server selects a prompt template that defines a fixed structure with variable placeholders. Server fills placeholders with case-specific values, such as disease names and scores, symptom descriptions, location, duration, and fever status.

Server assembles the text into a coherent instruction for the model. For example, server generates a prompt sentence such as:
“You are a medical assistant AI. A patient has submitted a skin image and answered a questionnaire.

Image analysis (CNN) suggests:
Possible conditions: allergic contact dermatitis (0.62), urticaria (0.25), eczema (0.10).
Image features: red maculopapular rash localized to the left forearm, irregular borders, no visible bleeding.

Questionnaire (converted to structured data):
Location: left forearm
Main symptom: red rash
Additional symptom: itching
Duration: 2 days
Fever: none
New medications or cosmetics: none reported

Based on this information, use your medical reasoning to:
1. List the most likely disease names in order of likelihood.
2. Explain briefly, in lay terms, why you think each condition is possible.
3. Indicate whether the patient should seek urgent care, non-urgent in-person consultation, or self-care with monitoring.

Respond in concise English suitable for a patient.”

The output of this step is a fully formed prompt sentence tailored to the case.

### Step 11:

Server calls the generative AI model with the prompt sentence and obtains diagnostic support text.

Server sends the constructed prompt sentence as input to the generative AI model via an inference interface. Server sets parameters such as maximum output length and sampling strategy to control the generation process. The generative AI model processes the prompt using its internal neural network architecture to produce a continuation of text. Server receives the generated text response, which typically contains possible disease names, reasoning explanations, and consultation urgency. The output of this step is a block of diagnostic support text linked to the case.

### Step 12:

Server parses and structures the diagnostic support text.

Server takes the generated text from Step 11 as input. Server analyzes the text to identify segments corresponding to disease names, reasoning content, and urgency levels, for example by searching for numbered lists or labeled sections requested in the prompt. Server may use simple pattern matching or a lightweight parser to extract elements such as “Condition 1: . . . ”, “Reason: . . . ”, and “Recommendation: . . . ”. Server converts this extracted information into a structured diagnostic support object with fields such as “candidate_diseases”, “explanations”, and “urgency_level”. The output of this step is structured diagnostic support information that can be combined with other data.

### Step 13:

Server evaluates severity by referencing severity information for candidate diseases.

Server uses the candidate disease names within the diagnostic support object as input keys to query a severity database. Server retrieves stored severity levels, typical risk categories, and associated flags such as “requires immediate attention” or “usually mild.” Server then computes a severity evaluation result for the case, for example by selecting the highest severity level among top candidates or by weighting severity with the generative model's likelihood ranking. Server encapsulates the resulting classification and any flags in an evaluation information object. The output of this step is evaluation information containing a severity evaluation result for the case.

### Step 14:

Server generates display information and transmits it to the terminal.

Server takes as input the structured diagnostic support information from Step 12 and the evaluation information from Step 13. Server formats this data into display information that includes summaries, detailed explanations, and visual indicators of severity and urgency.

Server may convert internal field names into user-friendly labels and may structure the content into sections or bullet points for easier reading on the terminal screen. Server serializes the display information into a response message and sends it to the terminal over the secure communication channel. The output of this step is a transmitted response containing user-ready diagnostic support and severity information.

### Step 15:

Terminal displays diagnostic support information and recommendations to the user.

Terminal receives the response message from the server as input. Terminal parses the display information and maps sections to graphical components on the screen, such as title labels, text fields, and icons indicating urgency. Terminal may allow the user to scroll through explanations and tap buttons to view additional details or facility recommendations if present. User views the possible disease names, explanations, and indicated urgency and can take action, such as deciding to visit a medical facility. The output of this step is the visual presentation of diagnostic support on the terminal and the user's understanding and potential subsequent actions based on that information.

### Application Example 2

Description follows regarding a flow of the specific processing in an Application Example 2. The units of the system described below are implemented by the data processing device 12 and the smart device 14. The data processing device 12 is called a “server” and the smart device 14 is called a “terminal”.

Conventional computer-implemented health support systems generally treat clinical text, image data, context, and emotional information as separate and loosely coupled channels. A typical architecture executes fixed diagnostic algorithms on symptom text, performs independent image classification on medical images, and optionally triggers rule-based alerts. In such architectures, interaction with a generative AI model is limited to simple, static question-and-answer exchanges, without systematic generation and orchestration of prompt sentences that reflect the system's internal state. As a result, the processor cannot fully exploit multimodal inputs or adapt inference behavior to changing risk conditions, and the overall system remains brittle, difficult to extend, and technically inefficient.

More specifically, known systems present several computer-technical drawbacks. First, the processor generally does not normalize and transform heterogeneous inputs (symptom text, numerical vital data, emotion indicators, and image data) into a unified structured representation that can be fed into downstream inference components in a consistent manner. This causes duplicated parsing logic, inconsistent feature extraction, and increased processing latency and memory usage, which degrades throughput on server hardware.

Second, conventional systems do not use the processor to systematically construct layered prompt sentences that programmatically control generative AI models over multiple stages of a pipeline. Prompt sentences, if used at all, are typically hand-crafted and static, and they do not incorporate machine-readable constraints, severity criteria, urgency rules, or image-analysis outputs. Consequently, the interaction between the processor and the generative AI model becomes opaque and non-deterministic, which impairs reproducibility, complicates debugging, and increases the risk of erroneous or unstable outputs.

Third, in many existing architectures, image analysis units and emotion analysis units operate in parallel but not as first-class inputs to a central decision engine. The processor often cannot integrate an image analysis result or an emotional state score as part of a unified inference context. This leads to underutilization of computing resources, increased network traffic due to redundant calls, and an inability to compute risk-aware severity and urgency metrics that adapt in real time based on multimodal evidence.

Fourth, the selection of medicines, the choice of medical facilities, and the orchestration of electronic payment are generally implemented as monolithic business-logic modules. These modules are not parameterized by the outputs of a generative AI model under explicit prompt control. As a result, the processor must rely on rigid rule sets and static databases, which makes it difficult to dynamically reconcile conflicting information, handle edge cases, or scale to new diseases and treatments without substantial code changes.

Fifth, known systems often lack an integrated mechanism by which the processor can trace and log the specific prompt sentences and AI outputs used at each step of the inference chain. Without such traceability, it is challenging to improve computational performance, refine prompt templates, or adjust model usage policies in response to observed failures. This limits the ability of the system to evolve and optimize its internal computer-implemented workflows.

Accordingly, there is a need for an improved computer-implemented system in which a processor is configured to (i) transform and unify heterogeneous health-related inputs into structured information, (ii) programmatically generate and sequence prompt sentences that drive a generative AI model as a controllable inference component at multiple stages, (iii) integrate text-based diagnosis, image-based analysis, and emotional-state assessment into severity and urgency evaluation, and (iv) use the resulting structured outputs to control selection of medicines, recommendation of medical facilities, and initiation of electronic payment. Such an arrangement improves the operation of the computer system itself, by enabling more efficient data flows, reducing duplicated computation, increasing determinism and observability of AI interactions, and providing a modular architecture that can be scaled and maintained more easily.

The specific processing by the specific processing unit 290 of the data processing device 12 in Application Example 2 is realized by the following means.

The present invention provides a server comprising a processor configured to receive information relating to a health condition from a user, analyze the received information to generate structured information including at least symptom data, numerical data, and emotional state data, generate a prompt sentence based on the structured information for causing a generative AI model to diagnose at least one candidate disease name, operate the generative AI model to obtain the at least one candidate disease name and a likelihood associated with the at least one candidate disease name, generate a prompt sentence based on the at least one candidate disease name and the structured information for causing the generative AI model or another inference unit to evaluate a severity of a symptom and an urgency of medical consultation, operate the generative AI model or the another inference unit to obtain the severity and the urgency, perform preprocessing on image information acquired from the user by using an image processing unit, input the preprocessed image information into a trained discrimination model to obtain an image analysis result, integrate the image analysis result with the at least one candidate disease name and the structured information, generate a prompt sentence based on the at least one candidate disease name, the severity, the urgency, and the image analysis result for causing the generative AI model to generate a diagnostic explanation and an action guideline for the user, operate the generative AI model to generate a natural language explanation message, and transmit the explanation message and notification information corresponding to at least one of the severity and the urgency to a terminal of the user. This enables the computer system to perform a unified and programmatically controlled multimodal inference pipeline, in which the processor orchestrates multiple calls to the generative AI model via explicitly constructed prompt sentences, thereby improving data consistency, reducing redundant computation, and enhancing the determinism, scalability, and maintainability of health-related diagnosis, severity assessment, and alert generation on the server.

The term “processor” refers to a hardware information processing unit, such as a central processing unit or an execution core of an information processing apparatus, that executes instructions to implement the functions of the system.

The term “user” refers to a person who provides health-related information to the system and who receives diagnostic explanations, recommendations, or notifications from the system.

The term “terminal” refers to an information processing device operated by the user, such as a portable communication device, that acquires input data from the user and communicates with the server.

The term “health condition” refers to a state of physical or mental health of the user, including current symptoms, medical history, vital signs, and related contextual information.

The term “information relating to a health condition” refers to data supplied by or on behalf of the user that describes the user's health condition, and may include symptom descriptions, numerical measurements, historical data, and emotional expressions.

The term “structured information” refers to health-related information that has been transformed by the processor into a predefined data format, such as a record or object with fields for symptom data, numerical data, emotional state data, and other attributes.

The term “symptom data” refers to elements of the structured information that describe observable or subjective manifestations of a health condition, such as pain, cough, fever, or rash.

The term “numerical data” refers to quantitative values associated with the health condition, such as body temperature, heart rate, blood pressure, or duration of symptoms.

The term “emotional state data” refers to data representing an estimated or reported emotional condition of the user, such as anxiety, fear, or calmness, and may be expressed as categories, scores, or other numerical indicators.

The term “generative AI model” refers to an automated inference model, implemented by a computing apparatus, that generates output data such as text or structured information from input data, by using machine-learned parameters.

The term “prompt sentence” refers to a textual input sequence supplied to the generative AI model, which specifies a task, conditions, or constraints for the generative AI model to perform, and which is programmatically generated by the processor.

The term “candidate disease name” refers to an identifier of a possible disease or disorder output by the generative AI model as a potential explanation of the user's health condition.

The term “likelihood” refers to a value associated with a candidate disease name that indicates a degree of probability, confidence, or relevance of that candidate disease name for the user's health condition.

The term “severity” refers to an evaluation value that represents the seriousness or impact level of a symptom or of a candidate disease on the user, and may be expressed by categories such as mild, moderate, or severe.

The term “urgency” refers to an evaluation value that represents how quickly medical consultation or intervention is recommended for the user, and may be expressed by qualitative or quantitative levels.

The term “another inference unit” refers to an inference component other than the generative AI model, such as a rule engine, a statistical model, or another machine learning model, which is executed by the processor to compute severity, urgency, or related values.

The term “image information” refers to data representing a visual state of the user or an object, such as a still image or a frame sequence, acquired by an image sensing device associated with the terminal or the system.

The term “image processing unit” refers to a functional module, implemented by hardware and software, that performs processing on image information, such as resizing, normalization, filtering, or feature extraction.

The term “trained discrimination model” refers to a machine-learned model, such as a classification model, that has been trained in advance to output an analysis result from input image information or other features.

The term “image analysis result” refers to data output by the trained discrimination model based on image information, such as classification labels, probabilities, or feature vectors associated with visual patterns.

The term “diagnostic explanation” refers to natural language text that describes a reason, basis, or interpretation of one or more candidate disease names, severities, or urgencies in a manner understandable to the user.

The term “action guideline” refers to natural language text that suggests one or more actions for the user to take, such as self-care, seeking medical consultation, or emergency response, based on the diagnostic explanation.

The term “explanation message” refers to a message containing at least the diagnostic explanation and optionally the action guideline, which is generated by using the generative AI model and transmitted to the terminal.

The term “notification information” refers to information indicating an alert, warning, or status related to at least one of the severity and the urgency, which is transmitted from the processor to the terminal.

The term “medicine information storage unit” refers to a storage resource, such as a database, that stores data representing medicines, including indications, contraindications, dosage information, and related attributes.

The term “candidate medicines” refers to medicines retrieved from the medicine information storage unit that satisfy one or more conditions based on the candidate disease names, severities, and user attribute information.

The term “recommended medicine information” refers to information identifying one or more medicines selected for recommendation by using the generative AI model, and including at least a name and a reason for recommendation.

The term “user attribute information” refers to data describing attributes of the user, such as age, sex, allergy history, chronic conditions, or residence region.

The term “electronic payment unit” refers to a functional module, implemented by software and hardware, that performs payment transaction processing for purchases of medicines or services using electronic payment methods.

The term “purchase process” refers to a sequence of operations in which payment authorization and settlement are performed for an order of a medicine, and in which an order record is generated or updated.

The term “provision arrangement” refers to processing by which the system initiates or manages fulfillment, delivery, or preparation of a medicine for the user, based on the purchase process.

The term “medical facility information storage unit” refers to a storage resource, such as a database, that stores data relating to medical facilities, including facility identifiers, locations, specialties, and capabilities.

The term “candidate medical facilities” refers to medical facilities retrieved from the medical facility information storage unit based on at least the candidate disease names, severities, urgencies, emotional state data, and user location information.

The term “diagnosis-related information” refers to information associated with the user's diagnosis, including the candidate disease names, the severity, the urgency, the emotional state data, and derived contextual data.

The term “recommended medical facility information” refers to information identifying one or more medical facilities that have been selected or ranked for recommendation by using the generative AI model.

The term “recommendation message” refers to a message that includes at least the recommended medical facility information and that is transmitted to the terminal to guide the user to an appropriate medical facility.

In an embodiment, a server cooperates with one or more terminals operated by a user to implement a multimodal health-support system. The server executes software modules on general-purpose computer hardware that includes at least one central processing unit, a main memory, a non-volatile storage device, a network interface, and optionally a graphics processing unit for accelerating machine learning inference. The terminal comprises an information processing device such as a smartphone, tablet, or wearable device, and includes a processor, a display, a microphone, a camera, a location sensor, and a communication interface.

The server stores executable instructions and data structures in the non-volatile storage device. The executable instructions define at least: an input normalization module, a natural language processing module, an emotion analysis module, an image preprocessing module, an image recognition module, a prompt generation module, a generative AI interface module, a severity and urgency evaluation module, a medicine selection module, a medical facility recommendation module, a notification control module, and a logging and optimization module. The server loads these instructions into the main memory and executes the instructions on the processor to implement the functions described below.

The terminal acquires health-related information from the user via a graphical user interface. The terminal displays input fields for symptoms, numeric values such as body temperature or heart rate, and medical history. The terminal acquires images of affected body parts through the camera, voice data describing the condition through the microphone, and position information through the location sensor. The terminal converts these inputs into structured messages, for example, in a key-value representation, and transmits the messages to the server using a secure communication protocol such as HTTPS.

The server receives the messages from the terminal and stores raw input data in a persistent data store. The server uses a natural language processing library, implemented for example by a general-purpose NLP toolkit, to process the text portion of the health information. The server executes tokenization, sentence segmentation, part-of-speech tagging, and named-entity recognition to identify symptom phrases, temporal expressions, numeric values, and mentions of diseases or medications. The server maps these entities into a structured information object with fields such as “symptom_list,” “duration,” “body_temperature,” “past_diseases,” “allergies,” and “reported_emotions.”

The server uses an emotion analysis module to derive emotional state data. When the user provides explicit emotional text (for example, “I feel very anxious and scared”), the server applies sentiment and emotion classification by executing a text classification model that outputs scores for categories such as “anxiety,” “fear,” “sadness,” and “calmness.” When the user provides voice or image data, the server extracts acoustic features such as pitch, energy, and spectral coefficients, and visual features such as facial landmarks and expression-related descriptors. The server inputs these features into a trained neural network classifier to estimate emotional state scores. The server merges these scores into the structured information object, for example, as numeric values in fields such as “anxiety_score” or “fear score.”

The server executes an image preprocessing module when image information is present. The server uses an image processing library to resize images to a predetermined resolution, normalize pixel values, optionally apply denoising filters, and crop regions of interest. The server then uses an image recognition module implemented as a trained convolutional neural network. In one embodiment, the network has multiple convolutional layers with rectified linear unit activations, pooling layers, and fully connected layers that output a vector of probabilities for skin-related or other disease categories. The server executes this network on a graphics processing unit to accelerate matrix multiplications and convolution operations.

The server stores the output of the image recognition module as an image analysis result, including the most likely category and associated probabilities.

The server integrates text-based semantic information, numeric values, emotional state data, and image analysis results in a unified internal record. This record uses a specific data structure with typed fields and identifiers that can be readily consumed by downstream modules. The server thereby avoids repeatedly re-parsing raw text or recomputing low-level features. This reduction of duplicate processing shortens execution time and decreases memory consumption, which improves the throughput of the server under high request load.

The server generates a prompt sentence for a generative AI model by using a prompt generation module. The server selects portions of the structured information and encodes them into a textual instruction. In one illustrative example, the server generates a prompt sentence such as:
“User symptoms: cough for 3 days, sore throat, temperature 37.8 degrees Celsius, no chronic diseases, no known allergies.
Task: As a medical assistant, list up to five possible disease names with likelihood scores between 0 and 1. Explain your reasoning briefly.”

In another example, when image information and text are both available, the server generates a prompt sentence such as:
“The image classifier suggests eczema with high probability for the attached skin image. The questionnaire text: ‘Itchy red rash on forearm for 2 days.’
Task: Considering both the classifier output and the text, provide the most likely disease name and classify the severity as mild, moderate, or severe. Explain in three sentences.”

The server also generates prompt sentences for emotion-aware severity estimation. In one example, the server generates a prompt sentence such as:
“Preliminary diagnosis: probable influenza.
Emotion scores: anxiety 0.9, fear 0.7.
Task: Considering symptoms and high anxiety, classify the overall risk as low, medium, or high, and state whether urgent medical attention is recommended. Provide a short explanation.”

The server additionally generates prompt sentences for medicine selection and medical facility recommendation. For medicine selection, the server can generate a prompt sentence such as:
“Diagnosis: common cold, mild.
User: 30 years old, no known allergies, no chronic diseases.
Task: Suggest three over-the-counter medications that can relieve cough and sore throat. For each, provide the medicine name, the type, and a one-sentence reason.”

For medical facility recommendation, the server can generate a prompt sentence such as:
“The user's main diagnosis is suspected pneumonia, severity moderate, urgency high.
Nearby facilities: Facility A, 2 km, general internal medicine, open; Facility B, 5 km, respiratory specialist, open; Facility C, 1 km, small clinic, no imaging equipment.
Task: Rank these facilities in order of suitability and briefly explain the ranking.”

The server supplies such prompt sentences to a generative AI model through the generative AI interface module. The generative AI model may be implemented as a transformer-based neural network with an encoder-decoder architecture or a decoder-only architecture. The model parameters are stored in memory and loaded onto the processing units. The server performs inference by applying multi-head self-attention operations, feed-forward layers, and layer normalization across token sequences representing the prompt sentences. The model generates output tokens sequentially, conditioned on the prompt sentences and any system-level instructions.

The server configures the generative AI model with medical-safety-specific instructions in a system prompt and defines output formats that are machine-readable, such as explicit labels or structured textual patterns. By using prompt sentences that encode explicit tasks, constraints, and desired formats, the server can systematically treat the generative AI model as a programmable inference component. This differs from merely substituting human judgment, because the processor orchestrates multiple model calls with precise task decompositions, integrates outputs with other machine-learned models, and applies non-trivial combination rules.

The server performs severity and urgency evaluation using both the outputs of the generative AI model and other inference units. For example, the server may apply threshold rules or a separate statistical model that maps disease likelihoods, body temperature, symptom duration, and emotional scores to discrete severity levels and urgency categories. The server stores these values in dedicated fields in the internal record. By separating the generative text generation from the numeric risk assessment, the server improves determinism and reduces the variance of results across repeated inferences.

The server uses the medicine selection module to query a medicine information storage unit. The medicine information storage unit may be implemented as a relational database with tables for active ingredients, indications, contraindications, dosage ranges, and regulatory classifications. The server applies filtering and join operations to obtain candidate medicines that match the diagnosed disease, satisfy allergy and contraindication constraints, and fall within allowed dosing ranges. The server then uses a prompt sentence to ask the generative AI model to rank or annotate these candidates. The server combines the database-derived structural constraints with the generative AI evaluation to produce recommended medicine information. This hybrid approach leverages both strict rule-based constraints and flexible natural language reasoning, resulting in improved precision and a reduction in unsafe or irrelevant recommendations.

The server uses the medical facility recommendation module to query a medical facility information storage unit that contains attributes such as facility identifiers, locations, specialties, emergency capabilities, and typical waiting times. The server retrieves candidates within a defined radius of the user's location and filters them by required specialties or equipment. The server then uses a prompt sentence to instruct the generative AI model to rank the facilities in view of the severity, urgency, and emotional state of the user. The server merges the ranking from the generative AI model with hard-coded priority rules (for example, always prioritize a facility capable of handling emergencies for high-urgency chest pain) to generate a robust recommendation list. This layered decision process uses the generative AI model as an adaptive component within a constrained and traceable selection pipeline.

The server controls a notification control module to transmit messages to the terminal. The server formats explanation messages generated by the generative AI model into compact payloads and transmits them to the terminal. The terminal displays the explanation messages, the severity level, the urgency guidance, the recommended medicines, and the recommended facilities. When severity or urgency exceeds predefined thresholds, the server generates high-priority notifications and may attach additional instructions, such as emergency contact information. The terminal can present visual alerts, audible alerts, or haptic feedback.

The server records the data flow, including prompt sentences, model outputs, intermediate feature vectors, and final decisions, in a logging system. The server uses these logs to measure processing latency, model response times, and error rates. The server can adjust prompt templates, threshold values, or model selection strategies to improve both the quality of outputs and the computational performance. For example, the server can switch to a smaller generative AI model for low-severity cases in order to reduce resource consumption, while keeping a larger model reserved for complex high-risk cases. This dynamic allocation contributes to improved overall throughput and reduced energy usage.

In one embodiment, the server uses supervised learning to train the image recognition model and the emotion recognition model. The server trains a convolutional neural network for image classification by minimizing a cross-entropy loss between predicted labels and ground truth disease labels for training images. The server applies stochastic gradient descent or an adaptive optimization algorithm to update model weights, uses mini-batching to improve computational efficiency, and applies data augmentation techniques such as random cropping, contrast adjustment, and rotation to improve generalization. The server similarly trains an emotion classifier on labeled voice or text datasets, using features such as spectrogram representations for audio or token embeddings for text. By explicitly specifying the training procedures, the system achieves robust and repeatable performance on heterogeneous real-world inputs.

In another embodiment, the server tailors the generative AI model to the health-support domain via fine-tuning or prompt-tuning techniques. The server collects de-identified training examples of prompt sentences and desired outputs, including disease lists, severity descriptions, and recommended actions. The server then trains a lightweight adapter layer or adjusts embedding vectors that are specific to the domain. This customization allows the generative AI model to respond more consistently to the system's prompt patterns, reducing hallucination rates and improving adherence to requested output formats. Consequently, the processor can rely on more stable and predictable model outputs, which directly improves system reliability.

In still another embodiment, the server employs a rule-based pre-filter on the structured information object before generating prompt sentences. For example, if the body temperature is below a first threshold and no severe symptoms are present, the server selects a “low-risk” prompt template that instructs the generative AI model to focus on self-care advice and routine consultation. If multiple high-risk indicators are present, the server selects a “high-risk” prompt template that emphasizes emergency triage decisions. By implementing these non-conventional prompt selection rules, the server reduces unnecessary computation and restricts the search space of the generative AI model, which both speeds processing and decreases the chance of inappropriate outputs.

The described system provides technical effects beyond merely automating human diagnostic reasoning. The architecture improves the internal operation of the computer system by introducing a unified structured information representation that consistently integrates text, numeric, image, and emotional data; by programmatically generating prompt sentences to control the generative AI model at multiple pipeline stages; by combining neural network outputs with deterministic rules and database queries; and by logging and optimizing model interactions based on system-level performance metrics. These features result in lower processing latency, improved diagnostic consistency, reduced communication overhead between modules, and a more maintainable and scalable design for health-related inference services.

Alternative implementations are also possible. For example, the server can distribute processing across multiple physical machines, with one machine dedicated to natural language and generative AI processing, another machine dedicated to image analysis, and a third machine managing databases and payment processing. The system can replace the neural network architectures with different network depths or layer configurations, such as residual blocks or attention-based image encoders. The emotion analysis module can use alternative feature sets or classification algorithms. The prompt generation module can support additional templates for languages other than English.

In each of these embodiments, however, the server continues to implement the core concepts: generation of structured information from heterogeneous inputs, generation and orchestration of prompt sentences to a generative AI model, integration of multimodal inference results, and technically grounded control of diagnosis explanation, medicine selection, facility recommendation, and notification in a way that improves the operation of the computer system itself.

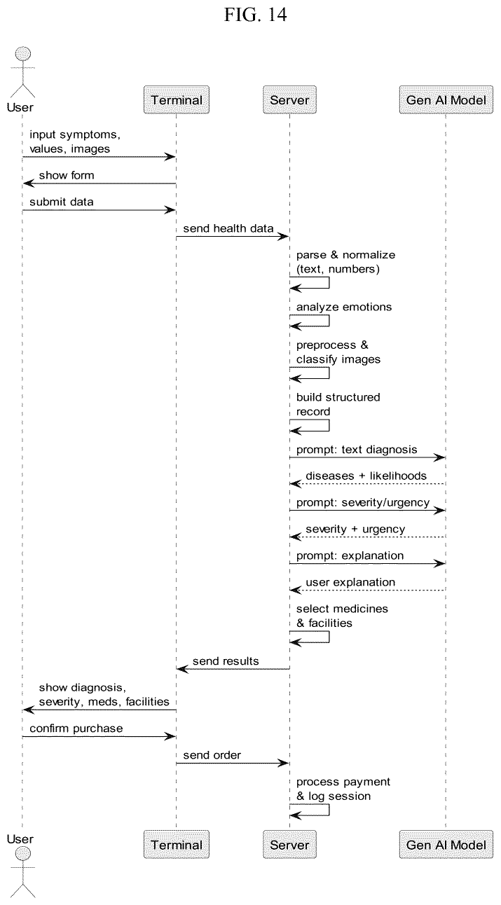

The following describes the processing flow using FIG. 14.

### Step 1:

User operates the terminal to input health-related information.

User enters symptom text (for example, “cough for 3 days, sore throat”), selects or enters numerical values (for example, body temperature), optionally enters medical history and allergy information, and may type an emotional description (for example, “I feel very anxious”). User may also capture one or more images of affected body parts using the camera of the terminal.

Input: raw user inputs including free-text symptoms, numerical values, optional history, emotional description, and images.

Output: a local data bundle on the terminal containing text fields, numeric fields, and image binary data.

Terminal converts the user's actions into internal data structures (for example, key-value pairs) and temporarily stores them in memory.

### Step 2:

Terminal optionally records voice input and converts it to text.

User speaks into the microphone to describe symptoms and feelings. Terminal records audio data using the operating system audio API and, if configured, invokes a speech recognition service or sends the audio to the server.

Input: raw audio waveform recorded from the microphone.

Output: transcribed text representing the user's spoken description.

Terminal, when using a local or remote speech recognizer, processes the waveform into acoustic features and maps them to text tokens, then appends the transcription to the symptom text field in the data bundle.

### Step 3:

Terminal packages and transmits the collected data to the server.

Terminal combines text, numerical values, images, and optional transcribed audio text into a message formatted for network transmission, and sends the message via a secure protocol to a server endpoint.

Input: internal data bundle containing user-entered and captured data.

Output: a network request containing serialized payload (for example, JSON for text/numerics and multipart for images) delivered to the server.

Terminal establishes a network connection and writes the serialized data into the request body, then initiates transmission.

### Step 4:

Server receives and stores the raw health-related data.

Server accepts the HTTP request from the terminal, parses headers and payload, and extracts text fields, numerical fields, and image data. Server assigns a session identifier and stores the raw data in persistent storage.

Input: network request from the terminal including user data.

Output: stored raw record associated with a session ID in a database or file store.

Server allocates memory buffers for the incoming data, validates the format, and executes database insert operations to store the raw record.

### Step 5:

Server performs text normalization and symptom extraction.

Server retrieves the raw text from storage and applies natural language processing routines to standardize and segment the text. Server performs tokenization, lowercasing where appropriate, sentence splitting, and removal of noise characters. Server then applies part-of-speech tagging and entity recognition to identify symptom-related expressions, durations, and mentioned diseases.

Input: raw text describing symptoms, history, and emotional expressions.

Output: structured textual features including a list of symptom tokens, associated attributes (for example, duration, intensity), and an initial list of candidate medical entities.

Server runs NLP algorithms that compute word embeddings or other features, matches them to symptom patterns, and populates a structured information object with fields such as “symptom_list” and “symptom_duration.”

### Step 6:

Server computes emotional state data from text and optional voice or image features.

Server analyzes any explicit emotional expressions in the text (for example, “very anxious,” “terrified”) using a text classification model to assign scores for emotion categories. If voice or facial image data is available, server extracts acoustic or visual features and feeds them into an emotion classifier to produce additional emotion scores.

Input: textual emotional descriptions, and optionally voice-derived or image-derived features.

Output: emotional state data represented as category labels and numeric scores (for example, anxiety_score, fear_score).

Server performs feature extraction (such as frequency analysis for voice, facial landmark detection for images) and then applies classifier inference to transform these features into quantitative emotion indicators, which are stored in the structured information object.

### Step 7:

Server preprocesses image data and performs disease-related image analysis.

Server loads the image data from storage, resizes each image to a predetermined resolution, normalizes pixel values, and optionally applies noise reduction filters. Server then inputs the processed image into a trained neural network, such as a convolutional classifier, to infer likely visual disease categories and probabilities.

Input: raw medical image data captured by the terminal.

Output: image analysis result including predicted categories (for example, type of rash) and associated probabilities.

Server executes matrix operations and convolutions on the image tensors, calculates activation maps in multiple layers, and derives a probability distribution over predefined disease labels, which is added to the internal record.

### Step 8:

Server constructs a unified structured information object.

Server merges the textual features, numerical values, emotional state data, and image analysis results into a single structured record. This record may include fields for symptom list, body temperature, emotion scores, image-based disease probabilities, and user attributes such as age or allergies.

Input: outputs from text processing, emotion analysis, image analysis, and raw numerical data.

Output: a unified structured information object representing the user's health status in a machine-readable format.

Server maps individual outputs to specific fields, resolves inconsistencies (for example, duplicate symptom entries), and serializes the object in an internal schema for further processing.

### Step 9:

Server generates a prompt sentence for an initial text-based diagnosis by the generative AI model.

Server selects relevant elements from the structured information object, such as symptom list, duration, and numerical values, and formats them into a natural language instruction for the generative AI model.

Input: structured information object without final diagnosis.

Output: a prompt sentence requesting candidate disease names and associated likelihoods.

Server concatenates fixed template phrases with dynamic content (symptom descriptions and numeric values), enforces an instruction structure, and produces a complete prompt sentence, for example:

“User symptoms: cough for 3 days, sore throat, temperature 37.8 degrees Celsius, no chronic diseases, no known allergies. Task: As a medical assistant, list up to five possible disease names with likelihood scores between 0 and 1, and briefly explain your reasoning.”

### Step 10:

Server invokes the generative AI model to obtain candidate disease names and likelihoods.

Server sends the constructed prompt sentence to the generative AI model interface, which feeds it into a neural network that computes attention weights over tokens and generates an output sequence predicting disease candidates.

Input: initial diagnostic prompt sentence.

Output: model-generated text describing candidate disease names and likelihood values.

Server processes tokens through multiple layers of the transformer architecture, accumulates logits for each vocabulary token, and decodes the sequence into text. Server then parses the generated text to extract disease names and corresponding likelihoods, which are inserted into the structured information object.

### Step 11:

Server generates a prompt sentence for severity and urgency evaluation.

Server uses the candidate disease names, likelihoods, and the structured information to build another instruction specifying that severity and urgency must be evaluated.

Input: candidate disease list, likelihoods, and structured information.

Output: a severity/urgency prompt sentence specifying evaluation criteria.

Server composes a textual instruction such as:
“Preliminary diagnosis: probable influenza with high likelihood. Symptoms: fever 38.5 degrees Celsius for 2 days, body aches, fatigue. Emotion scores: anxiety 0.9, fear 0.7. Task:
Classify overall severity as mild, moderate, or severe, classify urgency as low, medium, or high, and state briefly whether immediate medical attention is recommended.”

### Step 12:

Server evaluates severity and urgency using the generative AI model and/or another inference unit.

Server submits the severity/urgency prompt sentence to the generative AI model or passes numeric features into a separate statistical model to compute discrete severity and urgency levels.

Input: severity/urgency prompt sentence and associated numeric features.

Output: severity category, urgency category, and explanatory text.

Server either decodes the generative AI model output to identify written labels (for example, “moderate severity, high urgency”) or computes category boundaries with a dedicated model, and stores the resulting categories and explanation in the structured record.

### Step 13:

Server generates a combined prompt sentence for a user-facing diagnostic explanation.

Server aggregates the main candidate disease, severity, urgency, and image analysis findings into a new instruction asking the generative AI model to produce a concise explanation and action guideline in plain language.

Input: structured record including diagnosis, severity, urgency, and image analysis.

Output: explanatory prompt sentence for the generative AI model.

Server composes text such as:
“The primary suspected disease is influenza with moderate severity and high urgency. The user has fever, cough, and body aches, and the condition started 2 days ago. Task: Summarize this diagnosis for a non-expert user, explain in simple terms why influenza is suspected, and provide short advice on what the user should do next. Use clear and reassuring language.”

### Step 14:

Server obtains a natural language explanation message from the generative AI model.

Server sends the explanatory prompt sentence to the generative AI model and receives a generated explanation and guideline.

Input: explanatory prompt sentence.

Output: natural language explanation message suitable for display to the user.

Server runs the model inference, decodes output tokens into sentences, and verifies that essential items (disease name, severity, recommended action) are present. Server may post-process the explanation to enforce length limits or remove unsupported claims before storing it.

### Step 15:

Server selects candidate medicines based on the diagnosis and user attributes.

Server queries a medicine information storage unit using the primary disease, severity level, and user attributes such as allergies and age to retrieve medicines that satisfy clinical and regulatory constraints.

Input: disease name, severity, user attribute information.

Output: list of candidate medicines and associated metadata.

Server executes database search and filtering operations, computes intersections of indication sets and contraindication sets, and produces a subset of medicines that match the case.

### Step 16:

Server refines medicine recommendations using a generative AI model via a dedicated prompt sentence.

Server builds a prompt sentence that presents the candidate medicines and case details, asking the generative AI model to select and justify recommended medicines.

Input: candidate medicine list and structured case information.

Output: textual recommendation identifying selected medicines and reasons.

Server composes text such as:
“Diagnosis: common cold, mild. User: 30 years old, no known allergies, no chronic diseases. Candidate medicines: [list of medicines with indications]. Task: Select the most appropriate medicines to relieve cough and sore throat, and explain briefly why each is suitable.”

Server submits this prompt to the generative AI model, decodes the response, and maps recommended items back to specific medicine identifiers.

### Step 17:

Server selects candidate medical facilities using location and diagnosis data.

Server queries a medical facility information storage unit based on user location, disease type, severity, and urgency, retrieving facilities within a specified distance that have appropriate specialties and capabilities.

Input: location information, diagnosis, severity, urgency.

Output: list of candidate medical facilities with attributes such as distance and specialty.

Server performs spatial queries, calculates approximate distances, and filters out facilities that cannot provide required services.

### Step 18:

Server refines medical facility recommendations using a generative AI model via a ranking prompt sentence.

Server constructs a prompt sentence that presents the candidate facilities and case context and instructs the generative AI model to rank or select the most suitable facilities.

Input: candidate facility list and diagnosis-related information.

Output: textual ranking or selection of recommended medical facilities.

Server composes a prompt such as:
“The user's main diagnosis is suspected pneumonia, severity moderate, urgency high. Nearby facilities: Facility A, 2 km, general internal medicine, open; Facility B, 5 km, respiratory specialist, open; Facility C, 1 km, small clinic, no imaging equipment. Task: Rank these facilities in order of suitability for this user and explain your reasoning in a few sentences.”

Server processes the model's output to construct a structured recommendation list.

### Step 19:

Server prepares and sends response data, including explanation, severity, medicines, and facilities, to the terminal.

Server gathers the explanation message, severity and urgency values, recommended medicines, and facility recommendations, and composes a response payload.

Input: all computed outputs from previous steps.

Output: a response message containing diagnostic information and recommendations sent to the terminal.

Server serializes the data into a structured format, attaches any necessary metadata (timestamps, session ID), and transmits the payload to the terminal using the communication interface.

### Step 20:

Terminal displays diagnostic results and recommendations to the user.

Terminal receives the response from the server, parses the payload, and renders the explanation text, severity and urgency indicators, medicine proposals, and facility recommendations in the user interface.

Input: response message from the server.

Output: visual presentation on the display, and optionally audible or haptic alerts.

Terminal arranges UI elements, sets text labels, highlights urgent warnings, and presents actionable controls (for example, buttons to purchase medicines or view facility details).

### Step 21:

User reviews the information and optionally initiates medicine purchase or facility selection.

User reads the explanation and guidance, decides whether to purchase recommended medicines or to visit a suggested medical facility, and interacts with the terminal to confirm actions.

Input: displayed information and interactive controls.

Output: user choices represented as commands or confirmations sent back to the server.

User taps buttons for purchasing or for navigating to facility details, and the terminal encodes these actions into follow-up requests.

### Step 22:

Server processes purchase requests through an electronic payment unit.

Server receives a purchase confirmation and payment information from the terminal, communicates with an electronic payment service, and updates internal records based on the transaction result.

Input: purchase confirmation, selected medicines, and payment token.

Output: transaction status, updated order records, and confirmation or error messages.

Server sends transaction data to a payment gateway, awaits authorization, and, upon success, logs the order and may notify a fulfillment entity for delivery.

### Step 23:

Server logs processing details and updates performance and model interaction records.

Server records the prompt sentences, generative AI outputs, intermediate decisions, latency metrics, and outcome data in a logging system for future analysis.

Input: all intermediate and final processing artifacts associated with the session.

Output: persistent log entries enabling performance monitoring and system optimization.

Server writes structured log records, indexes them by session and component, and may compute aggregate statistics to adjust thresholds, refine prompt templates, or choose between different generative AI model variants in future sessions.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

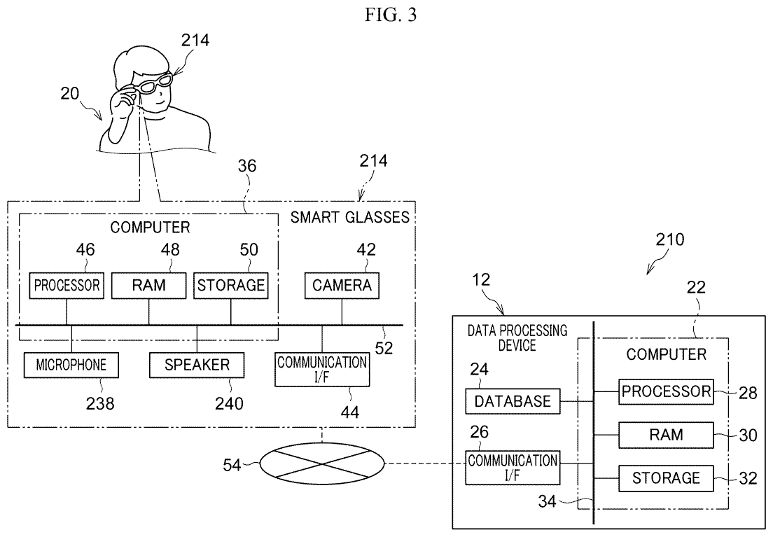

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52.

The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

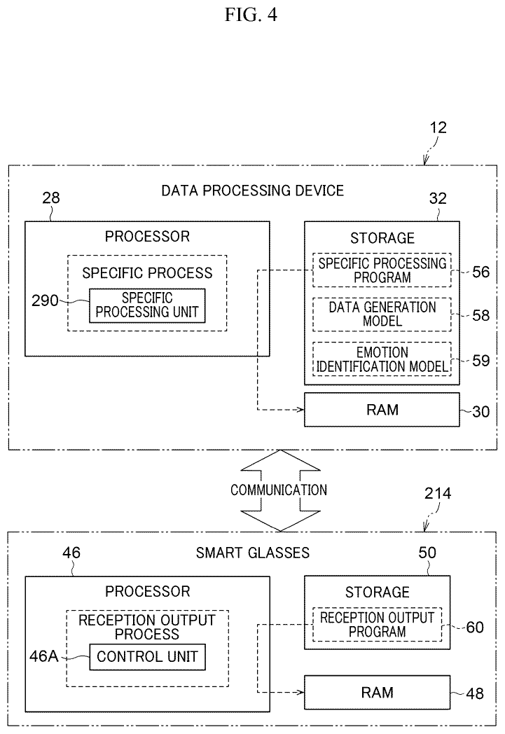

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### Third Exemplary Embodiment

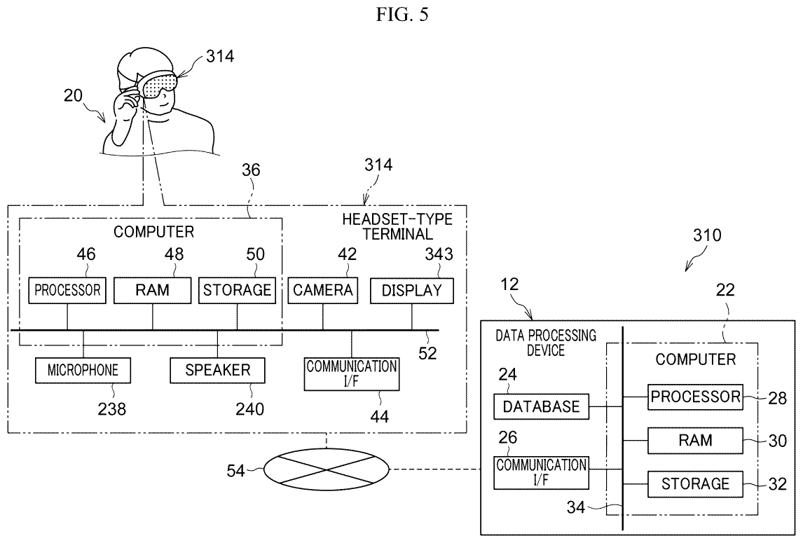

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

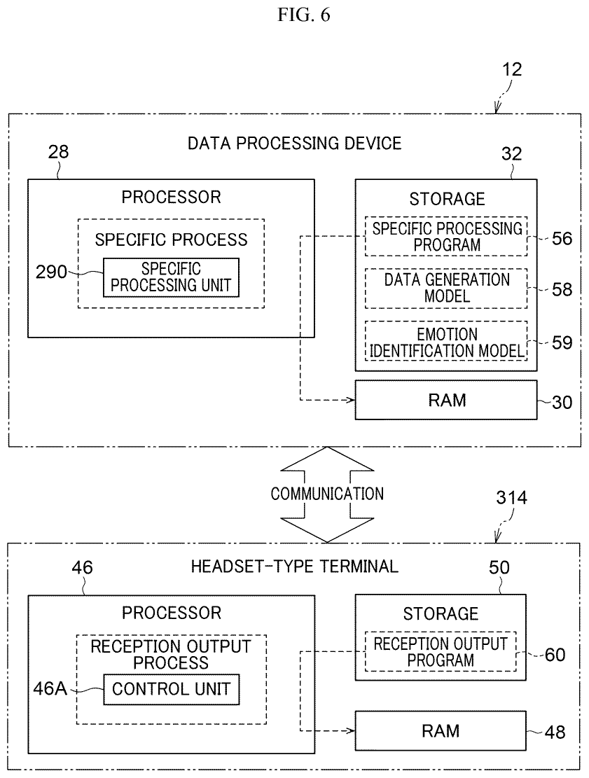

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

### Fourth Exemplary Embodiment

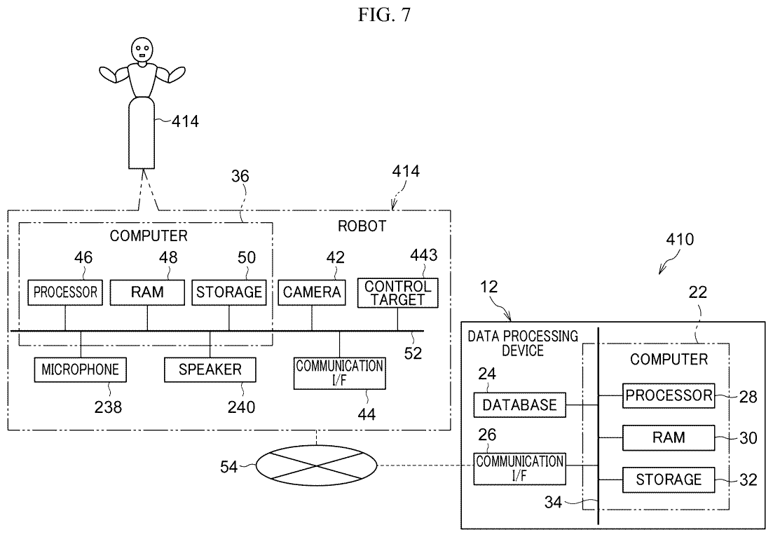

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

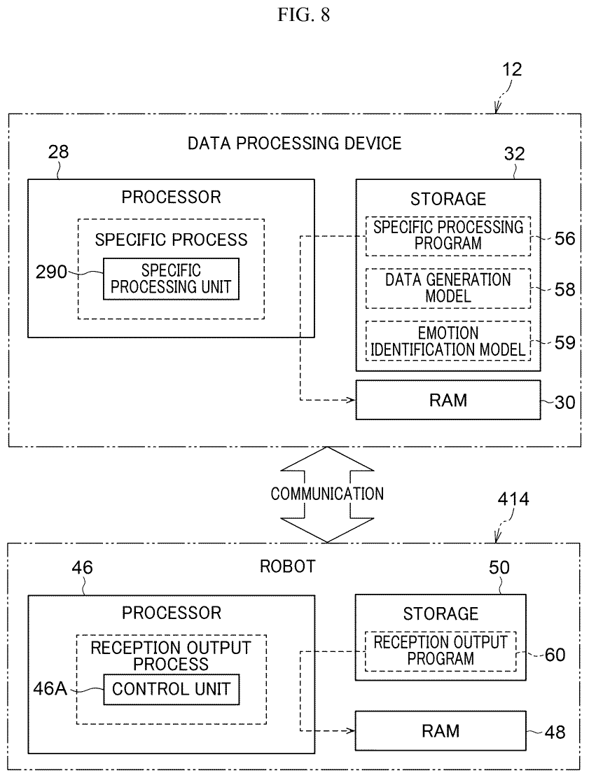

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

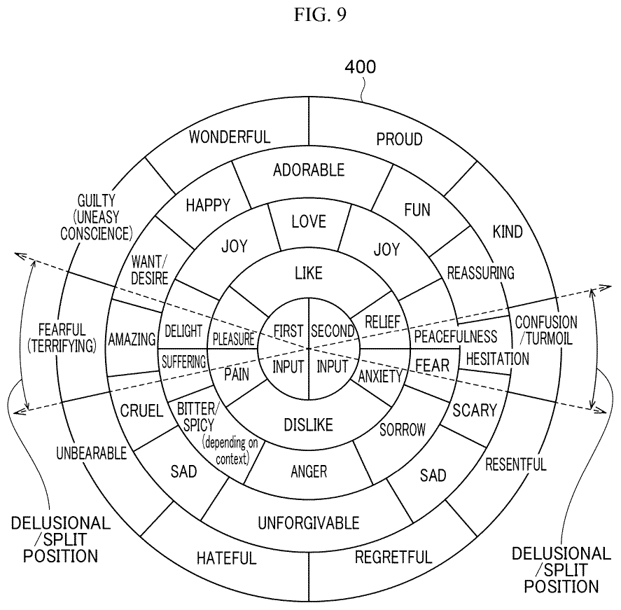

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University). Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more” and “want to know more” is experienced.

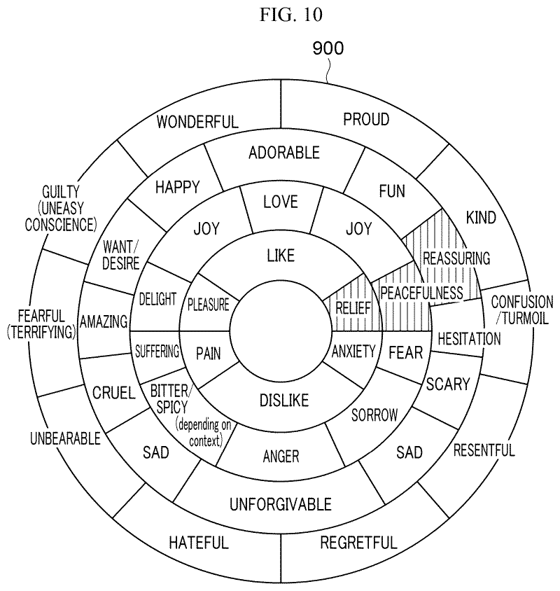

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (Saas).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### (Supplementary 1)

A system comprising a processor,
wherein the processor is configured to
receive health status information from a user terminal and convert the health status information into structured data and store the structured data,
generate a prompt sentence described in natural language based on the structured data including information on symptoms, body temperature, and medical history, and input the prompt sentence into a generative AI model so as to cause the generative AI model to analyze the structured data and to obtain candidate disease names and explanation information, refer to an information storage device that stores severity information associated with the candidate disease names, acquire severity for each of the candidate disease names, and
determine overall severity based on the candidate disease names and the severity information, and
generate diagnosis result information to be presented to a user based on the candidate disease names, the severity information, and the explanation information, and transmit the diagnosis result information to the user terminal.

### (Supplementary 2)

The system according to supplementary 1,
wherein the processor is configured to
generate the prompt sentence so as to include a structured output condition that instructs the generative AI model to output the candidate disease names and the explanation information in a machine-readable format, and to parse the candidate disease names and the explanation information output in the machine-readable format to extract the candidate disease names.

### (Supplementary 3)

The system according to supplementary 1,
wherein the processor is configured to
refer to an information storage device that stores information on a plurality of medical service providers based on the candidate disease names and the severity information, select a medical service provider suitable for the user, and include recommendation information comprising a result of the selection in the diagnosis result information.

### Application Example 1

### (Supplementary 1)

A system comprising a processor,
wherein the processor is configured to
receive information relating to a health condition from a user,
preprocess the information relating to the health condition to convert numerical information and character information into a predetermined format,
generate a prompt sentence for input to an analysis generative AI model by using the preprocessed information, and instruct analysis by the generative AI model,
specify a possible disease name and a severity of the disease on a basis of an analysis result output from the generative AI model,
refer to an information storage unit in which medical expense payment plans corresponding to the specified severity are defined, and extract a medical expense payment plan applicable to the user,
generate payment plan presentation information for presenting the extracted medical expense payment plan to the user, and
transmit the payment plan presentation information to a user terminal.

### (Supplementary 2)

The system according to supplementary 1,
wherein the processor is configured to
further include a function of integrating the analysis result by the analysis generative AI model and a disease estimation result by a prediction learning model stored in the information storage unit, and determining the disease name and the severity on a basis of the integrated result.

### (Supplementary 3)

The system according to supplementary 1,
wherein the processor is configured to
generate, on a basis of the disease name, the severity, and the medical expense payment plan, a prompt sentence to be input to an explanation generative AI model for generating an explanation text relating to the medical expense payment plan, and include an output result acquired from the explanation generative AI model in the payment plan presentation information.

### Example 2

### (Supplementary 1)

A system comprising a processor and a storage device,
wherein the processor is configured to
receive, via a secure communication protocol, image data and text data acquired from an information processing terminal operated by a user, and convert the received image data into analysis data by performing preprocessing including standardization and noise reduction, input the preprocessed image data into an image recognition artificial intelligence model based on convolution operations, extract feature data from the image data, and generate candidate disease information corresponding to the image data by comparing the feature data with record information stored in the storage device and including symptom information, on the basis of a similarity measure,
perform text analysis based on natural language processing on the text data, and convert the text data into structured data including at least symptom content, onset location, elapsed time, accompanying symptoms, and medical history information,
integrate the candidate disease information and the structured data, generate case information summarizing an image analysis result and interview information, and generate a prompt sentence configured to cause a generative artificial intelligence model to generate diagnosis candidates and explanatory information on the basis of the case information,
input the prompt sentence to the generative artificial intelligence model and cause the generative artificial intelligence model to generate diagnostic support information including at least candidate disease names, reasoning content, and a consultation urgency level based on the case information,
refer to severity information stored in the storage device and associated with the candidate disease names included in the diagnostic support information, and generate evaluation information including a severity evaluation result, and convert the diagnostic support information and the evaluation information into display information in a format transmittable to the information processing terminal, and transmit the display information to the information processing terminal.

### (Supplementary 2)

The system according to supplementary 1,
wherein the processor is configured to
refer to the storage device including position information of the user and attribute information of medical service providers, on the basis of the candidate disease information, the structured data, and the evaluation information, select an appropriate medical service provider on the basis of at least a medical department, available facilities, and consultation priority, and generate, for the generative artificial intelligence model, a prompt sentence configured to cause the generative artificial intelligence model to generate a recommendation message including a selection result and a consultation reason.

### (Supplementary 3)

The system according to supplementary 1,
wherein the processor is configured to
refer to the storage device including pharmaceutical information on the basis of the candidate disease information and the structured data, extract candidate pharmaceutical information, generate a prompt sentence configured to cause the generative artificial intelligence model to generate guidance information including candidates of pharmaceuticals suitable for the symptoms of the user and precautions for use, by using the candidate disease information, the structured data, and the candidate pharmaceutical information as input conditions, and input the prompt sentence to the generative artificial intelligence model.

### Application Example 2

### (Supplementary 1)

A system comprising a processor,
wherein the processor is configured to
receive information relating to a health condition from a user,
analyze the received information and generate structured information including at least symptom data, numerical data, and emotional state data,
generate a prompt sentence, based on the structured information, for causing a generative AI model to diagnose at least one candidate disease name, and operate the generative AI model to obtain the at least one candidate disease name and a likelihood associated with the at least one candidate disease name,
generate a prompt sentence, based on the at least one candidate disease name and the structured information, for causing the generative AI model or another inference unit to evaluate a severity of a symptom and an urgency of medical consultation, and operate the generative AI model or the another inference unit to obtain the severity and the urgency, perform preprocessing on image information acquired from the user by using an image processing unit, input the preprocessed image information into a trained discrimination model to obtain an image analysis result, and integrate the image analysis result with the at least one candidate disease name and the structured information,
generate a prompt sentence, based on the at least one candidate disease name, the severity, the urgency, and the image analysis result, for causing the generative AI model to generate a diagnostic explanation and an action guideline for the user, and operate the generative AI model to generate a natural language explanation message, and
transmit the explanation message and notification information corresponding to at least one of the severity and the urgency to a terminal of the user.

### (Supplementary 2)

The system according to supplementary 1,
wherein the processor is configured to
search a medicine information storage unit based on the at least one candidate disease name, the severity, and user attribute information to extract candidate medicines,
generate a prompt sentence, based on the candidate medicines and the structured information, for causing the generative AI model to select appropriate medicine candidates and reasons therefor, and operate the generative AI model to obtain recommended medicine information, and
operate an electronic payment unit based on the recommended medicine information and payment information of the user to perform a purchase process and provision arrangement for a medicine.

### (Supplementary 3)

The system according to supplementary 1,
wherein the processor is configured to
search a medical facility information storage unit based on the at least one candidate disease name, the severity, the urgency, the emotional state data, and location information of the user to generate a list of candidate medical facilities,
generate a prompt sentence, based on the list of candidate medical facilities and diagnosis-related information, for causing the generative AI model to rank or narrow down the candidate medical facilities, and operate the generative AI model to obtain recommended medical facility information, and
generate a recommendation message including the recommended medical facility information and transmit the recommendation message to the terminal of the user.

What is claimed is:

1. A system comprising:
circuitry configured to:
receive, via a communication interface coupled to a packet-switched network, input data from a terminal device;
convert the input data into structured data and store the structured data in a storage device;
construct a first prompt data structure including the structured data and an instruction sequence directing a generative neural network model to analyze the structured data and output candidate classification labels and associated explanation data in a machine-readable format;
transmit the first prompt data structure to the generative neural network model and parse response data to extract the candidate classification labels;
retrieve, from the storage device, grading data associated with each of the candidate classification labels, and determine an overall grading value on the basis of the candidate classification labels and the grading data; and
generate result data including the candidate classification labels, the grading data, and the explanation data, and transmit the result data to the terminal device via the packet-switched network.

2. The system according to claim 1, wherein the circuitry converts the input data into the structured data by applying a natural language processing operation that detects entity tokens in the input data and maps each entity token to a field in a predetermined data schema, the predetermined data schema including at least a condition description field, a numerical measurement field, and a temporal field.

3. The system according to claim 2, wherein the circuitry normalizes values in the numerical measurement field to a standardized range and validates the entity tokens against a reference vocabulary stored in the storage device, and constructs the first prompt data structure to include both the normalized values and the validated entity tokens.

4. The system according to claim 3, wherein the condition description field includes symptom content data and accompanying symptom data, the numerical measurement field includes body temperature data, the temporal field includes an onset time and an elapsed duration, and the predetermined data schema further includes a history field storing prior condition records associated with the terminal device.

5. The system according to claim 4, wherein the input data represents health condition information of a user, the candidate classification labels represent disease name candidates, the grading data represents severity information associated with each disease name candidate, and the overall grading value represents a composite severity assessment determined by aggregating the severity information across the disease name candidates.

6. The system according to claim 1, wherein the generative neural network model comprises a transformer architecture including a token embedding layer, a positional encoding mechanism, a plurality of self-attention layers, and feedforward sublayers, and the instruction sequence specifies a structured output condition requiring the response data to include the candidate classification labels and the explanation data as delimited fields in the machine-readable format.

7. The system according to claim 6, wherein the circuitry parses the response data by identifying field delimiters specified in the structured output condition, extracts a classification field as the candidate classification labels, extracts a reasoning field as the explanation data, and validates each candidate classification label against a set of permissible labels stored in the storage device.

8. The system according to claim 7, wherein the circuitry further integrates the candidate classification labels with a prediction result obtained from a prediction model stored in the storage device, the prediction model receiving the structured data as input and outputting a probability distribution over the set of permissible labels, and the circuitry determines final classification labels by combining the candidate classification labels from the generative neural network model with the probability distribution from the prediction model.

9. The system according to claim 1, wherein the input data further includes image data received from the terminal device, and the circuitry preprocesses the image data by performing at least standardization and noise reduction to generate analysis image data.

10. The system according to claim 9, wherein the circuitry inputs the analysis image data to an image recognition model based on convolution operations, extracts feature data from the analysis image data, and generates candidate information by comparing the feature data with record information stored in the storage device on the basis of a similarity measure.

11. The system according to claim 10, wherein the circuitry integrates the candidate information generated from the image data with the structured data generated from text-based input data, generates case summary data combining an image analysis result and the structured data, and constructs the first prompt data structure to include the case summary data as context for the generative neural network model.

12. The system according to claim 1, wherein the circuitry constructs a second prompt data structure including the candidate classification labels, the overall grading value, and an instruction sequence directing the generative neural network model to select one or more remediation items from a remediation database stored in the storage device on the basis of the candidate classification labels and the overall grading value.

13. The system according to claim 12, wherein the remediation database stores records associated with one or more nearby service facilities, each record including item availability data, and the circuitry selects the one or more remediation items by filtering the records on the basis of the candidate classification labels and an availability status, and incorporates the selected remediation items into the result data transmitted to the terminal device.

14. The system according to claim 1, wherein the circuitry constructs a third prompt data structure including the candidate classification labels, the overall grading value, and location data associated with the terminal device, and an instruction sequence directing the generative neural network model to recommend one or more service facilities on the basis of the candidate classification labels, the overall grading value, and the location data.

15. The system according to claim 14, wherein the one or more service facilities are medical institutions, the circuitry filters medical institution records stored in the storage device on the basis of the location data and specialty fields matching the candidate classification labels, and the result data transmitted to the terminal device includes a recommendation message identifying the filtered medical institutions and associated contact data.

16. The system according to claim 1, wherein the circuitry further inputs the input data to an emotion analysis model and obtains emotion data indicating an emotional state associated with the input data, and constructs a fourth prompt data structure including the candidate classification labels, the explanation data, and the emotion data, and an instruction sequence directing the generative neural network model to adjust a complexity level and a reassurance level of the explanation data on the basis of the emotion data.

17. The system according to claim 16, wherein the circuitry retrieves, from the storage device, expense plan data associated with the overall grading value, constructs a fifth prompt data structure including the expense plan data and an instruction sequence directing the generative neural network model to generate an expense explanation text, and incorporates the expense explanation text into the result data transmitted to the terminal device.

18. A system comprising:
circuitry configured to:
receive, via a communication interface coupled to a packet-switched network, input data including text data and image data from a terminal device;
apply a natural language processing operation to the text data to detect entity tokens and map the entity tokens to fields in a predetermined data schema to generate structured data, and store the structured data in a storage device;
preprocess the image data by performing standardization and noise reduction, input the preprocessed image data to an image recognition model based on convolution operations to extract feature data, and generate candidate information by comparing the feature data with record information stored in the storage device on the basis of a similarity measure;
integrate the candidate information and the structured data to generate case summary data, and construct a first prompt data structure including the case summary data and an instruction sequence directing a generative neural network model comprising a transformer architecture with a token embedding layer, a positional encoding mechanism, a plurality of self-attention layers, and feedforward sublayers to output candidate classification labels and explanation data in a machine-readable format;
parse response data from the generative neural network model to extract the candidate classification labels, retrieve grading data associated with each candidate classification label from the storage device, and determine an overall grading value;
input the text data to an emotion analysis model and obtain emotion data indicating an emotional state, and construct a second prompt data structure including the candidate classification labels, the explanation data, and the emotion data, the second prompt data structure directing the generative neural network model to adjust a complexity level of the explanation data on the basis of the emotion data; and
generate result data including the candidate classification labels, the grading data, the explanation data, and the overall grading value, and transmit the result data to the terminal device via the packet-switched network.

19. The system according to claim 18, wherein the circuitry integrates the candidate classification labels with a prediction result obtained from a prediction model that receives the structured data as input and outputs a probability distribution over permissible labels, and determines final classification labels by combining the candidate classification labels with the probability distribution.

20. A method performed by circuitry of a server coupled to a packet-switched network via a communication interface, the method comprising:
receiving, via the communication interface, input data from a terminal device;
converting the input data into structured data and storing the structured data in a storage device;
constructing a first prompt data structure including the structured data and an instruction sequence directing a generative neural network model to analyze the structured data and output candidate classification labels and associated explanation data in a machine-readable format;
transmitting the first prompt data structure to the generative neural network model and parsing response data to extract the candidate classification labels;
retrieving, from the storage device, grading data associated with each of the candidate classification labels, and determining an overall grading value on the basis of the candidate classification labels and the grading data; and
generating result data including the candidate classification labels, the grading data, and the explanation data, and transmitting the result data to the terminal device via the packet-switched network.
